(19) United States
(12) Patent Application Publication
(10) Pub. No.: US 2017/0103166 A1
(43) Pub. Date: Apr. 13, 2017

(54) WEARABLE DEVICE FOR PROVIDING SERVICE ACCORDING TO MEASUREMENT OF BLOOD ALCOHOL LEVEL AND MANAGEMENT SERVER THEREFOR

(71) Applicant: SK PLANET CO., LTD., Gyeonggi-do (KR)

(72) Inventors: Sungoh OH, Seoul (KR); Sunghyun YOON, Seoul (KR)

(73) Assignee: SK PLANET CO., LTD., Gyeonggi-do (KR)

(21) Appl. No.: 15/290,620

(22) Filed: Oct. 11, 2016

(30) Foreign Application Priority Data
Oct. 13, 2015 (KR) 10-2015-0142645
Oct. 27, 2015 (KR) 10-2015-0149614
Oct. 30, 2015 (KR) 10-2015-0152110

(51) Int. Cl. G06F 19/00; A61B 5/024; A61B 5/18; G06Q 30/02; A61B 5/053; A61B 5/08; A61B 5/00; A61B 5/01; A61B 5/11

(52) U.S. Cl. CPC G06F 19/322; A61B 5/01; A61B 5/024; A61B 5/18; A61B 5/11; A61B 5/0537; A61B 5/08; A61B 5/6803; A61B 5/681; A61B 5/0022; A61B 5/6824; G06Q 30/0239

(57) ABSTRACT

Disclosed herein is a wearable device. A blood alcohol level of a user is measured in real time, and information about the location of the user is measured. When the blood alcohol level of the user is a predetermined value or more, information about a coupon or point which may be used at a store within a predetermined range may be requested from a management server or a chauffeur service may be requested.

### CROSS REFERENCE TO RELATED APPLICATION

The present application claims the benefit of Korean Patent Application No. 10-2015-0152110 filed in the Korean Intellectual Property Office on Oct. 30, 2015, Korean Patent Application No. 10-2015-0142645 filed in the Korean Intellectual Property Office on Oct. 13, 2015 and Korean Patent Application No. 10-2015-0149614 filed in the Korean Intellectual Property Office on Oct. 27, 2015, the entire contents of which are incorporated herein by reference.

### BACKGROUND OF THE INVENTION

1. Technical Field

The present invention relates to a wearable device for providing a service according to the measurement of a blood alcohol level and, more particularly, to a technology in which a blood alcohol level of a user is measured in real time, information about the location of the user is measured, and a management server requests information about a coupon or point which may be used at a store within a predetermined range or requests a chauffeur service if the blood alcohol level of the user is a predetermined value or more.

2. Description of the Related Art

A wearable device literally refers to a device which may be worn on the human body, and includes various types of devices, such as glasses, a watch, a bracelet, shoes, a ring, a belt, a band, a necklace, a headset, and clothing, depending on a part on which the device is worn. Today, such a wearable device is coming into wide use. From among the wearable devices, the wearable glasses, the wearable watch, and the wearable band which are very closely related to the human life and can be easily worn form the most product group.

The wearable device is also called a smart device. The reason for this is that the wearable device needs to be initially driven by user input, but once the wearable device is driven, it can automatically generate various types of additional information and provide them to a user. For example, if a user who has worn a wearable watch wants to measure his or her own heart rate, the wearable watch can measure the heart rate of the user as soon as the user presses a heart rate measurement input button, can generate various types of information based on the measured heart rate, and can provide them to the user. Recently, many wearable devices that do not need to be initially driven by user input are appearing. For example, a wearable band can continue to measure the heart rate of a user although it is not initially driven by user input. In the future, there is a very good possibility that a wearable device will be developed in such a way not to be initially driven by user input. The reason for this is that convenience in device driving is improved if user input is not required.

The origin of various types of additional information provided from such a wearable device to a user includes pieces of information measured from the user. For example, the pieces of information may include the heart rate, blood pressure, mental state, weight, body temperature, respiration volume, number of steps, matter of concern, current location, etc of a user. Accordingly, sensors for measuring various types of information from a user need to be mounted on a wearable device. The more the type of sensor is increased, the more information is measured from a user. Accordingly, a heart rate measurement sensor, a Global Positioning System (GPS) sensor and/or a number-of-steps measurement sensor tend to be essentially mounted on a recent wearable device. Research continues to be carried out to dispose various sensors in a limited space within a wearable device.

A wearable device can measure information from a user through sensors mounted thereon, can generate various types of additional information based on the measured information, and can provide them to the user. For example, if blood pressure of a user is too high as a result of the measurement of the blood pressure, information about a medicine capable of lowering the blood pressure to a proper level may be provided to the user. Alternatively, the current location of the user may be measured, and information about a nearby store or commodities related to a matter of concern of the user may be provided to the user. Such additional information may be provided through a function embedded in a wearable device itself, but it is impossible for a single wearable device to provide all pieces of additional information to different users. Accordingly, recently, an application installed on a wearable device is also developed. In this case, the application may be considered to be a kind of application program, and may function to generate various types of additional information based on information measured through a function embedded in a wearable device itself and to provide them. In the above example, the provision of information about a medicine capable of lowering blood pressure to a proper level or information about a nearby store or commodities related to a matter of concern of a user can be provided by an application. That is, whenever a new application is developed, additional information which may be provided to a user through a wearable device is inevitably diversified. The development of an application may be considered to be a companion that steps forward along with the development of a wearable device itself.

Recently, to construct a system capable of settlement through a wearable device is emerging as a main matter of concern in a related industry. For example, if a near field communication (NFC) function is included in a wearable device and a user's credit card is registered with an application, the user can perform settlement through the wearable device although the user does not take his or her purse out of his or her pocket. That is, a mobile wallet function can be performed by replacing a conventional wallet. However, there are many problems to be solved due to many problems related to security, such as personal information and the approval of credit card payment.

Furthermore, in a related art, a method of a user blowing out the steam of breath is used to measure a blood alcohol level. Accordingly, it is difficult for a user to measure his or her blood alcohol level in real time while drinking if he or she is not conscious that he or she has to measure the blood alcohol level. For this reason, a user frequently drinks too much while being not conscious although he or she has an alcohol concentration measurement device.

### PRIOR ART DOCUMENT

(Patent Document 1) Korean Patent Application Publication No. 10-2012-0081332 entitled “Drunkometer system combined with multimedia advertising system”

### SUMMARY OF THE INVENTION

In an embodiment of the present invention, a blood alcohol level of a user is measured in real time, and thus the user can refrain from overdrinking because he or she can continue to check his or her blood alcohol level.

Furthermore, an embodiment of the present invention is directed to the provision of a coupon and points available for a hangover after drinking to a user.

Furthermore, an embodiment of the present invention is directed to providing a chauffeur service to a user although the user does not call the chauffeur service in such a manner that a management server checks the time when the user performs payment at a food store along with a blood alcohol level of the user and checks the time when the chauffeur service is required.

Furthermore, an embodiment of the present invention is directed to the measurement of an accurate quantity of motion of a user by analyzing the quantity of motion with consideration taken of body information, such as the age, sex, height, weight and body composition of the user in addition to a motion of the user in measuring the user's quantity of motion.

Furthermore, an embodiment of the present invention is directed to the provision of fitness equipment information, sporting goods information, food information and a coupon which enable a user to have a required amount of muscle, body fat, etc. based on information, such as the body composition, quantity of motion, etc. of the user.

Furthermore, in an embodiment of the present invention, a user can conveniently and frequently perform self-diagnosis without a burden regardless of people of all ages and both sexes by conveniently measuring his or her body composition for himself or herself.

Furthermore, an embodiment of the present invention is directed to helping a user to have a balanced body by checking the amount of activity of each part, performing a comparison between the amounts of muscle and body fat of parts, and notifying the user of an unbalanced part.

Furthermore, an embodiment of the present invention is directed to the provision of a method and apparatus, which are capable of measuring the composition ratio of sweat of a user through a wearable device and providing useful information to the user.

Furthermore, an embodiment of the present invention is directed to the provision of a method and apparatus, which are capable of measuring the composition ratio of sweat of a user, providing useful information, and also obtaining a specific marketing effect.

Technical objects to be achieved by the present invention are not limited to the objects, and they may include various technical objects within a range evident to those skilled in the art from the following description.

In an aspect of the present invention, a service providing system according to the measurement of a blood alcohol level may include a wearable device configured to measure a blood alcohol level of a user in a predetermined cycle, to measure information about the location of the user, and to request information about a coupon or point which may be used at a store within a predetermined range from a management server when the blood alcohol level of the user is a predetermined value or more and the management server configured to monitor a real-time blood alcohol level of the user received from the wearable device, to send a warning to the wearable device when the monitored blood alcohol level is the predetermined value or more, and to send a coupon or point which may be used at a store within a predetermined range from the location of the wearable device to the wearable device when the blood alcohol level of the user is the predetermined value or more.

In this case, the service providing system according to the measurement of a blood alcohol level may further include a user device configured to receive information about a coupon or point which may be used at a store within a predetermined range from the location of the wearable device from the management server when the blood alcohol level of the user is the predetermined value or more and to perform payment.

Furthermore, the management server may send a chauffeur call query to the wearable device when the blood alcohol level of the user is the predetermined value or more, and may send information about the location and destination of the user of the wearable device to a chauffeur driver device within a predetermined range from the location of the wearable device when a chauffeur call request is received from the wearable device.

A wearable device according to an embodiment of the present invention may include an alcohol concentration measurement unit configured to measure a blood alcohol level of a user in a predetermined cycle, a location information measurement unit configured to measure information about the location of the user, a coupon request unit configured to request information about a coupon or point which may be used at a store within a predetermined range from a management server when the blood alcohol level of the user is a predetermined value or more, and a communication unit configured to transmit and receive information about the blood alcohol level of the user and information about the location of the user to and from an external apparatus over a communication network.

In this case, the alcohol concentration measurement unit may measure the blood alcohol level based on sweat discharged from the body of the user, may measure the blood alcohol level based on a change of a body temperature of the user, or may measure the blood alcohol level based on a change of the heart rate of the user.

The wearable device according to an embodiment of the present invention may further include a chauffeur request unit configured to query the user as to whether a chauffeur call request is to be transmitted to the management server when the blood alcohol level of the user is the predetermined value or more.

Furthermore, the communication unit may send the settlement signal of the user to the management server when the user performs settlement at the food store when the alcohol concentration of the user is the predetermined value or more.

Furthermore, the alcohol concentration measurement unit may store the blood alcohol level of the user, may check the drinking habit of the user, and may notify the user of the possibility of a disease attributable to the drinking habit of the user.

The wearable device according to an embodiment of the present invention may further include a display unit configured to display the blood alcohol level of the user in real time and to display a warning message when the blood alcohol level of the user is the predetermined value or more.

A management server according to an embodiment of the present invention may include a database configured to store user information and information about a coupon or point which may be used at a stored, an alcohol concentration monitoring unit configured to monitor a real-time blood alcohol level of a user received from a wearable device and to send a warning to the wearable device when the blood alcohol level is a predetermined value or more, a location information unit configured to check the location of the user of the wearable device by receiving information about the location of the wearable device, a coupon providing unit configured to send a coupon or point which may be used at a store within a predetermined range from the location of the wearable device to the wearable device when the blood alcohol level of the user is the predetermined value or more, and a server communication unit configured to perform the transmission and reception of pieces of information along with an external apparatus over a communication network.

In this case, the coupon providing unit may divide the blood alcohol level of the user into predetermined values and differentially supply the coupon or point.

The management server according to an embodiment of the present invention may further include a chauffeur call unit configured to send a chauffeur call query to the wearable device when the blood alcohol level of the user is the predetermined value or more and to send information about the location and destination of the user of the wearable device to a chauffeur driver device within a predetermined range from the location of the wearable device when a chauffeur call request is received from the wearable device.

In this case, the chauffeur call unit may send a chauffeur call query to the wearable device when a settlement signal is received from the wearable device.

The management server according to an embodiment of the present invention may further include a health management unit configured to store the blood alcohol level of the user in the database, to check the drinking habit of the user, and to notify the user of the possibility of a disease attributable to the drinking habit of the user.

A service providing method according to the measurement of a blood alcohol level in accordance with an embodiment of the present invention may include the steps of (a) measuring, by a wearable device, a blood alcohol level of a user and sending, the blood alcohol level to a management server; (b) measuring, by the wearable device, information about the location of the user and sending the location information to the management server; (c) monitoring a real-time blood alcohol level of the user by receiving information about the real-time blood alcohol level from the management server; (d) checking, by the management server, the location of the user of the wearable device by receiving information about the location of the wearable device; and (e) sending, by the management server, a coupon or point which may be used at a store within a predetermined range from the location of the wearable device to the wearable device when the blood alcohol level of the user is a predetermined value or more.

In this case, the service providing method may further include the steps of (f1) sending, by the management server, a chauffeur call query to the wearable device when the blood alcohol level of the user is the predetermined value or more and (f2) sending, by the management server, information about the location and destination of the user of the wearable device to a chauffeur driver device within a predetermined range from the location of the wearable device when a chauffeur call request is received from the wearable device, after the step (d).

In an aspect of the present invention, a method for providing, by a wearable device, a service according to the measurement of a blood alcohol level in accordance with an embodiment of the present invention may include the steps of (a) measuring a blood alcohol level of a user; (b) measuring information about the location of the user; and (c) requesting information about a coupon or point which may be used at a store within a predetermined range from a management server when the blood alcohol level of the user is a predetermined value or more.

In this case, the step (a) includes measuring the alcohol concentration based on at least one of the sweat, body temperature, and heart rate of the user.

The method for providing, by a wearable device, a service according to the measurement of a blood alcohol level in accordance with an embodiment of the present invention may further include the step of (d) requesting a chauffeur call from the management server when the blood alcohol level of the user is the predetermined value or more after the step (b).

In some embodiments, the method for providing, by a wearable device, a service according to the measurement of a blood alcohol level in accordance with an embodiment of the present invention may further include the step of (e) sending the settlement signal of the user to the management server when the user performs payment at a food store when the alcohol concentration of the user is the predetermined value or more, after the step (b).

The method for providing, by a wearable device, a service according to the measurement of a blood alcohol level in accordance with an embodiment of the present invention may further include the step of (f) outputting the blood alcohol level of the user in real time and outputting a warning message when the blood alcohol level of the user is the predetermined value or more, after the step (b).

A method for providing, by a management server, a service according to the measurement of a blood alcohol level in accordance with an embodiment of the present invention may include the steps of (a) monitoring a real-time blood alcohol level of a user by receiving information about the real-time blood alcohol level from a wearable device; (b) checking the location of the user of the wearable device by receiving information about the location of the wearable device; and (c) sending a coupon or point which may be used at a store within a predetermined range from the location of the wearable device to the wearable device when the blood alcohol level of the user is a predetermined value or more.

In this case, the step (c) may include dividing the blood alcohol level of the user into predetermined values and differentially supplying the coupon or point.

Furthermore, the method for providing, by a management server, a service according to the measurement of a blood alcohol level in accordance with an embodiment of the present invention may further include the steps of (d1) sending a chauffeur call query to the wearable device when the blood alcohol level of the user is the predetermined value or more; and (d2) sending information about the location and destination of the user of the wearable device to a chauffeur driver device within a predetermined range from the location of the wearable device when a chauffeur call request is received from the wearable device, after the step (b).

Furthermore, the step (d1) may include sending the chauffeur call query to the wearable device when a settlement signal is received from the wearable device.

In another aspect of the present invention, a wearable device according to an embodiment of the present invention may include a measurement module configured to measure the body composition or motion of a user, a control module configured to calculate the quantity of motion based on the measured motion of the user, and a communication module configured to send information about the measured body composition or quantity of motion to a management server and to receive feedback information for the transmitted information from the management server.

Furthermore, the control module may calculate the quantity of motion by taking into consideration at least one of pieces of body information, such as the height, weight, body composition, and sex of the user.

Furthermore, the measurement module may measure the amount of muscle or body composition of each pat of the user and notify the user of an unbalanced part.

The measurement module may include a body composition measurement sensor configured to measure the body composition by measuring a resistance value generated when an electric current flows into the body of the user and a motion sensor configured to measure at least one of the muscle motion, heartbeat, and respiration volume of the user.

In this case, the feedback information may include at least one of fitness equipment information, sporting goods information, food information, and coupon information corresponding to information about the body of the user.

A management server according to an embodiment of the present invention may include a database configured to store and update user information or feedback information, a server communication unit configured to receive information about the body composition or quantity of motion of a user measured by a wearable device and to send information selected by a server control unit to the wearable device, and the server control unit configured to select feedback information from the database based on the received information about the body composition or quantity of motion.

Furthermore, the server control unit may recommend at least one of food, fitness equipment, and sporting goods which are suitable to achieve a predetermined target body measurement value based on information about the current body of the user.

Furthermore, the server control unit may update information about the body composition of each user in real time whenever information about the body composition of the user is received from each wearable device.

A system for measuring a body composition and providing feedback information according to an embodiment of the present invention may include a wearable device configured to measure the body composition or motion of a user, to calculate the quantity of motion based on the measured motion of the user, and to send information about the measured body composition or quantity of motion to a management server, and to receive feedback information for the transmitted information from the management server, and the management server configured to store and update user information or feedback information, to receive information about the body composition or quantity of motion of the user from the wearable device, to select feedback information from a database based on the received information about the body composition or quantity of motion, ad to send the selected information to the wearable device.

In this case, in an embodiment of the present invention, the system for measuring a body composition and providing feedback information may further include a user terminal configured to store the information about the body composition and quantity of motion measured by the wearable device or the feedback information received from the management server and to output the information.

A service providing method according to an embodiment of the present invention may include measuring, by a wearable device, the body composition or motion of a user; calculating, by the wearable device, the quantity of motion based on the measured motion of the user and sending information about the measured body composition or quantity of motion to a management server; receiving, by the management server, information about the measured body composition or quantity of motion of the user from the wearable device; selecting, by the management server, feedback information from a database based on the received information about the body composition or quantity of motion and sending the selected information to the wearable device; and receiving, by the wearable device, the feedback information from the management server.

In this case, the service providing method may further include updating, by the management server, information about the body composition of each user in real time whenever information about the body composition of each user is received from each wearable device.

Furthermore, the feedback information may include at least one of user information, fitness equipment information, sporting goods information, food information, and coupon information.

Furthermore, the service providing method may further include sending, by the wearable device, information about the measured body composition and quantity of motion to a user terminal; and sending, by the management server, the feedback information to the user terminal.

A service providing method for measuring, by a wearable device, a body composition and providing feedback information according to an embodiment of the present invention may include measuring, by a measurement module, information about the body composition or motion of a user; calculating, by a control module, the quantity of motion based on the measured motion of the user; and sending, by a communication module, information about the measured body composition or quantity of motion to a management server and to receive feedback information for the transmitted information from the management server.

In this case, calculating the quantity of motion may include calculating the quantity of motion by taking into consideration at least one of pieces of body information, such as the height, weight, body composition, and sex of the user.

Furthermore, measuring the body composition or motion of the user may include measuring the amount of muscle or body fat of each part of the user and notifying the user of an unbalanced part.

Furthermore, the feedback information may include at least one of fitness equipment information, sporting goods information, food information, and coupon information corresponding to information about the body of the user.

Measuring the body composition or motion of the user may include measuring the body composition by measuring a resistance value generated when an electric current flows into the body of the user and measuring at least one of the muscle motion, heartbeat, and respiration volume of the user.

A service providing method for measuring, by a management server, a body composition and providing feedback information according to an embodiment of the present invention may include storing, by a database, user information or feedback information; receiving, by a server communication unit, information about the body composition or quantity of motion of the user measured by a wearable device; selecting, by a server control unit, feedback information from the database based on the received information about the body composition or quantity of motion; and sending, by the server communication unit, the information selected by the server control unit to the wearable device.

In this case, selecting the feedback information may include recommending at least one of food, fitness equipment, and sporting goods which are suitable to achieve a predetermined target body measurement value based on information about the current body of the user.

The service providing method may further include updating information about the body composition of each user in real time whenever the information about the body composition of the user is received from each wearable device.

In an aspect of the present invention, a service providing method according to a first embodiment of the present invention includes (a) detecting, by a sensor unit, sweat secreted from the body of a user who has worn a wearable device, (b) measuring, by the sensor unit, a composition ratio of the detected sweat and storing the measured composition ratio in a memory unit along with the current location of the user as first information, (c) sending, by a communication unit, the first information stored in the memory unit to an information providing apparatus, (d) receiving, by the communication unit, second information which is a result of the analysis of the first information from the information providing apparatus, and (e) outputting, by a control unit, the second information to one or more of a display unit and a speaker unit.

Furthermore, the wearable device may include any one of a smart watch, smart glasses, and a smart band.

Furthermore, if the wearable device is the smart watch, the sensor unit may be mounted on one or more of the back of the main body of the smart watch and the inside of the strap of the smart watch.

Furthermore, if the wearable device is the smart glasses, the sensor unit may be mounted on one or more of the nose pad, inside of the temple, and inside of the end piece of the smart glasses.

Furthermore, the second information may include information about a commodity to be recommended to the user in relation to the composition ratio of the sweat of the user which is included in the first information.

Furthermore, the second information may further include information about a place where a commodity to be recommended to the user is handled, which is a closest to the current location of the user.

Furthermore, the service providing method according to the first embodiment of the present invention may further include the step of (d-1) storing, by the control unit, the received second information in the memory unit after step (d).

A management server according to a second embodiment of the present invention includes a commodity information unit configured to store commodities to be recommended to a user and information about the commodities in a list form according to composition that belongs to the compositions of sweat and that has a high composition rate, a map unit configured to store an updatable map, and a second information generation unit configured to generate second information by analyzing first information received from a communication unit.

Furthermore, the information about the commodity may further include the name, price, and handling place of the commodity, and may further include one or more of the type of membership point applied, a discount coupon applied, and a saved coupon applied.

Furthermore, the second information generation unit may generate the second information by analyzing the first information and selecting one or more pieces of information that belong to the information about the commodities stored in the commodity information unit and that comply with the results of the analysis from the list.

Furthermore, the second information generation unit may generate the second information by selecting the place where one or more commodities selected from the list is handled, which is included in information about the one or more commodities, from the map unit.

Furthermore, the placed selected by the second information generation unit may be the place that is included in the first information and that is the closest to the current location of the user.

### BRIEF DESCRIPTION OF THE DRAWINGS

FIG. 1 is a reference diagram illustrating a process for implementing a service providing system according to the measurement of a blood alcohol level in accordance with an embodiment of the present invention.

FIG. 2 is a diagram showing the configuration of a service providing system according to the measurement of a blood alcohol level in accordance with an embodiment of the present invention.

FIG. 3 is a diagram showing the configuration of a function block of a wearable device according to an embodiment of the present invention.

FIG. 4 is a diagram showing the configuration of a function block of a management server according to an embodiment of the present invention.

FIG. 5 is a flowchart illustrating a service providing method according to the measurement of a blood alcohol level in accordance with an embodiment of the present invention.

FIG. 6 is a flowchart illustrating a method for providing, by the wearable device, a service according to the measurement of a blood alcohol level in accordance with an embodiment of the present invention.

FIG. 7 is a flowchart illustrating a method for providing, by the management server, a service according to the measurement of a blood alcohol level in accordance with an embodiment of the present invention.

FIG. 8 is a diagram showing the configuration of a function block of a system for measuring a body composition and providing feedback information according to an embodiment of the present invention.

FIG. 9 is a diagram showing the configuration of a function block of a wearable device according to an embodiment of the present invention.

FIG. 10 is a diagram showing the configuration of a function block of a management server according to an embodiment of the present invention.

FIG. 11 is a flowchart illustrating a service providing method according to an embodiment of the present invention.

FIG. 12 is a flowchart illustrating a method for measuring, by the wearable device, a body composition and providing feedback information according to an embodiment of the present invention.

FIG. 13 is a flowchart illustrating a method for measuring, by the management server, a body composition and providing feedback information according to an embodiment of the present invention.

FIG. 14 is a reference diagram illustrating the state in which the measurement module of the wearable device according to an embodiment of the present invention has been attached to an arm.

FIG. 15 is a reference diagram illustrating the state in which the measurement module of the wearable device according to an embodiment of the present invention is composed of a body composition measurement sensor and a motion sensor.

FIG. 16 is a diagram showing major elements of the wearable device.

FIG. 17 is a flowchart of a service providing method according to a first embodiment of the present invention.

FIG. 18 is a diagram showing the state in which a sensor unit has been mounted on the inside of the strap of a smart watch.

FIG. 19 is a diagram showing the state in which the sensor unit has been mounted on the back of the main body of a smart watch.

FIG. 20 is a diagram showing the state in which second information has been output through a smart watch.

FIG. 21 is a diagram showing the state in which second information has been output through a smart phone owned by a user who has worn a smart band.

FIG. 22 is a diagram showing the state in which the sensor unit has been mounted on the nose pad, inside of the temple, and inside of the end piece of smart glasses.

FIG. 23 is a diagram showing the state in which second information has been output through smart glasses.

FIG. 24 is a diagram showing the state in which second information has been output through smart glasses owned by a user who has worn a smart band.

FIG. 25 is a diagram showing the state in which a sensor unit has been mounted on the inside of the strap of a smart band.

FIG. 26 is a flowchart illustrating an example in which a service providing method according to a first embodiment of the present invention is performed through the smart band.

FIG. 27 is a diagram showing the configuration of a management server according to a second embodiment of the present invention.

FIG. 28 is a diagram showing a commodity information unit in which commodities to be recommended to a user and information about the commodities have been listed and stored according to compositions that belong to the remaining compositions forming sweat and that have a higher composition ratio.

FIG. 29 is a diagram showing a map stored in a map unit in which the places where commodities to be recommended to a user are handled have been marked.

FIG. 30 is a diagram showing an example in which a second information generation unit selects the closest place where a corresponding commodity is handled from the current location of a user.

### DETAILED DESCRIPTION

Hereinafter, some embodiments of the present invention are described in detail with reference to the exemplary drawings. The embodiments are provided so that those skilled in the art may easily understand the technical spirit of the present invention and the present invention is not restricted by the embodiments. A detailed description of the known functions and constructions will be omitted if it is deemed to make the gist of the present invention unnecessarily vague. The details of the objects and technical configurations of the present invention and acting effects thereof will be more clearly understood from the following detailed description based on the accompanying drawings. Hereinafter, embodiments of the present invention are described in detail with reference to the accompanying drawings.

Embodiments disclosed in this specification should not be interpreted as limiting or used to limit the scope of the present invention. It is evident to those skilled in the art that a description including the embodiments of this specification has various applications. Accordingly, unless otherwise defined by the claims, some embodiments described are illustrative for better understanding, and the scope of the present invention is not intended to be restricted by the embodiments.

Function blocks illustrated in the drawings and described hereunder are only examples of possible implementations. In other implementations, different functional blocks may be used without departing from the spirit and scope of the detailed description. Furthermore, one or more functional blocks of the present invention are illustrated as separate blocks, but one or more of the functional blocks of the present invention may be a combination of various hardware and software elements for executing the same function.

Furthermore, it should be understood that an expression that some elements are “included” is an expression of an “open type” and the expression simply denotes that the corresponding elements are present, but does not exclude additional elements. Furthermore, contents represented in the accompanying drawings have been diagrammed in order to easily describe the embodiments of the present invention, and the contents may be different from drawing forms that are actually implemented. It is to be noted that in assigning reference numerals to elements in the drawings, the same reference numerals denote the same elements throughout the drawings even in cases where the elements are shown in different drawings.

Furthermore, when it is said that one element is “connected” or “coupled” to the other element, it should be understood that one element may be directly connected or coupled” to the other element, but a third element may exist between the two elements.

Furthermore, expressions, such as “the first” and “the second”, are expressions used to only distinguish a plurality of elements from one another, and do not limit the sequence of the elements or other characteristics.

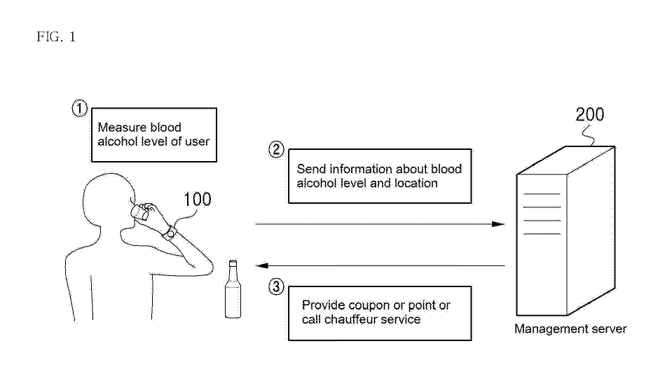

FIG. 1 is a reference diagram illustrating a process for implementing a service providing system according to the measurement of a blood alcohol level in accordance with an embodiment of the present invention.

Referring to FIG. 1, a wearable device 100 according to an embodiment of the present invention is mounted on the body of a user, and measures a blood alcohol level of the user in real time through means, such as sweat, a body temperature and/or the heart rate, and frequently notifies the user of the blood alcohol level so that the user does not drink too much. A management server 200 also monitors a blood alcohol level of the user in real time, checks the location of the user, and supplies a coupon or point which may be used at a store close to the location of the user. Furthermore, the management server 200 may call a chauffeur service if it is determined that the user requires the chauffeur service.

In this specification, in general, the “management server 200” may not be equipped with all the functions of a server which communicates with a client, and is simply named a server because it receives information from an external apparatus and provides information to the external apparatus. Accordingly, the management server may collectively refer to all devices, which communicate with the wearable device 100 or which are included in the wearable device 100 and monitor a blood alcohol level of a user.

The state of mind and body depending on a blood alcohol level of a person is as follows. If the blood alcohol level is 0.05%, feeling becomes a little better, but there is no great change. If the blood alcohol level is 0.1%, a body response time increases and self-control is weakened. If the blood alcohol level is 0.2%, the sense of sight or the sense of hearing and a language function are reduced. If the blood alcohol level is 0.3%, it is difficult to keep a body balance and the ability to perceive is significantly reduced. If the blood alcohol level is 0.45, a mind becomes confused like dizziness and walking is impossible. If the blood alcohol level is 0.65%, a person may lose his or her consciousness or may fall into a comatose state. Meanwhile, in Korea, driving in the state in which the blood alcohol level is 0.05% or more is classified as drunken driving.

Accordingly, in an embodiment of the present invention, when a user drinks, the user can refrain from overdrinking because he or she can objectively determine his or her drinking state based on an index through the wearable device 100. Accordingly, an accident, etc. attributable to excessive drinking can be prevented.

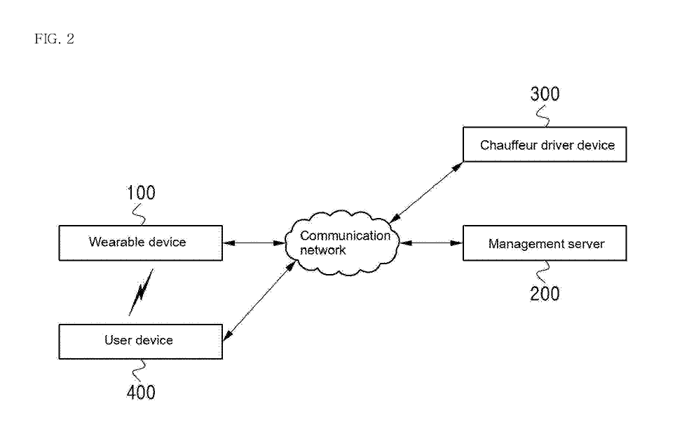

FIG. 2 is a diagram showing the configuration of a service providing system according to the measurement of a blood alcohol level in accordance with an embodiment of the present invention.

Referring to FIG. 2, the service providing system according to the measurement of a blood alcohol level in accordance with an embodiment of the present invention includes the wearable device 100 and the management server 200. More specifically, the service providing system includes the wearable device 100 configured to measure a blood alcohol level of a user in a predetermined cycle, to measure information about the location of the user, and to request information about a coupon or point which may be used at a store within a predetermined range from the management server 200 when the measured blood alcohol level of the user is a predetermined value or more and the management server 200 configured to monitor a real-time blood alcohol level of the user received from the wearable device 100 and to send a warning to the wearable device 100 or to send a coupon or point which may be used at a store within a predetermined range from the location of the wearable device 100 to the wearable device 100 when the monitored blood alcohol level is a predetermined value or more.

The wearable device 100 literally refers to a device which may be worn on the human body, and includes various types of devices, such as glasses, a watch, a bracelet, shoes, a ring, a belt, a band, a necklace, a headset, and clothing, depending on a part on which the device is worn.

The wearable device 100 used in an embodiment of the present invention is worn on the body of a user, and may measure a blood alcohol level of the user in real time through means, such as sweat, a body temperature and/or the heart rate, may frequently notify the user of the blood alcohol level so that the user does not drink too much, and may operate in conjunction with the management server 200.

The management server 200 may monitor a real-time blood alcohol level of the user received from the wearable device 100, and may send a warning to the wearable device 100 or to send a coupon or point which may be used at a store within a predetermined range from the location of the wearable device 100 to the wearable device 100 when the monitored blood alcohol level is a predetermined value or more.

For example, when the blood alcohol level of the user exceeds 0.05%, the management server 200 may send a warning message indicating that driving is impossible to the wearable device 100. When the blood alcohol level exceeds 0.1%, the management server 200 may provide a hangover beverage coupon which may be used at a store close to the location of the user or a food coupon or point for drinking to relieve the handover, which may be used the next day. In addition, when the alcohol concentration is 0.45%, the management server 200 may send a warning message indicating that consciousness may be lost if the user drinks further to the wearable device 100.

A service providing system according to the measurement of a blood alcohol level according to another embodiment of the present invention may further include a user device 400 configured to receive information about a coupon or point which may be used at a store within a predetermined range from the location of the wearable device 100 from the management server 200 when a blood alcohol level of a user is a predetermined value or more and to perform settlement.

In this case, the user device 400 may be a user terminal, for example. The user terminal includes a portable phone, a Personal Digital Assistant (PDA), a cellular phone, a Personal Communication Service (PCS) phone, a Global System for Mobile (GSM) phone, a Wideband CDMA (W-CDMA) phone, a CDMA-2000 phone, a Mobile Broadband System (MBS) phone, a Portable Multimedia Player (PMP), a Mobile Internet Device (MID), a smart phone, a desktop, a tablet PC, a note book, a net book and an information communication device which may be applied to various wired/wireless environments. The user device 400 may refer to a portable small-sized device, but may refer to a mobile communication terminal if it includes a camcorder, a laptop computer or the like. Accordingly, in an embodiment of the present invention, the user device 400 is not specifically limited thereto. Furthermore, a chauffeur driver device 300 to be described later may also be a portable terminal or a communication device mounted on a taxi, for example.

The management server 200 may send a chauffeur call query to the wearable device 100 when the blood alcohol level of the user is the predetermined value or more, and may send information about the location and destination of the user of the wearable device 100 to the chauffeur driver device 300 within a predetermined range from the location of the wearable device 100 when it receives a chauffeur call request from the wearable device 100.

The wearable device 100, the management server 200, the chauffeur driver device 300, and the user device 400 may transmit and receive information over a communication network.

The communication network includes a base station controller, a base station transmitter, a relay station, etc. The base station controller functions to relay a signal between the base station transmitter and a switching station. The communication network supports both a synchronous method and an asynchronous method. Accordingly, in the case of the synchronous method, a Base Station Transmission System (BTS) may become a transmission/reception base station transmitter and a Base Station Controller (BSC) may become a transmission/reception base station controller. In the case of the asynchronous method, a Radio Transceiver Subsystem (RTS) may become the transmission and reception base station transmitter, and a Radio Network Controller (RNC) may become the transmission and reception base station controller. The communication network according to an embodiment of the present invention is not limited thereto, and may collectively refer to a GSM network other than a CDMA network and networks which may be used in the access network of a next-generation mobile communication system to be developed in the future.

The communication network may further include an access point. The access point is a small base station, such as a femto or pico base station chiefly installed in a building. The femto or pico base station is classified depending on how many the wearable devices 100 can be accessed in terms of the classification of a small base station. Furthermore, the access point includes a short-range communication module for performing short-range communication, such as Wi-Fi, along with the wearable device 100. The short-range communication may be performed in accordance with various standards, such as Radio Frequencies (RF) and an ultra-wideband communication (UWB) including Bluetooth communication, Zigbee communication, infrared rays communication (IrDA), a Ultra High Frequency (UHF), and a Very High Frequency (VHF), in addition to Wi-Fi. The access point may extract the location of a data packet, may designate the best communication route for the extracted location, and may transfer the data packet to a next device, for example, the wearable device 100 along the designated communication route. The access point may be shared by several lines in a common network environment.

The access point is basically divided into a fixed type access point and a mobile type access point. The fixed type access point may include a router, a repeater, a relay station, etc. The mobile type access point may include the bridge product of a specific manufacturer, such as KT's Egg. Such a mobile type access point may read a reception-side address from transmission-side information while guaranteeing free mobility, may designate the most appropriate communication route, and may send it.

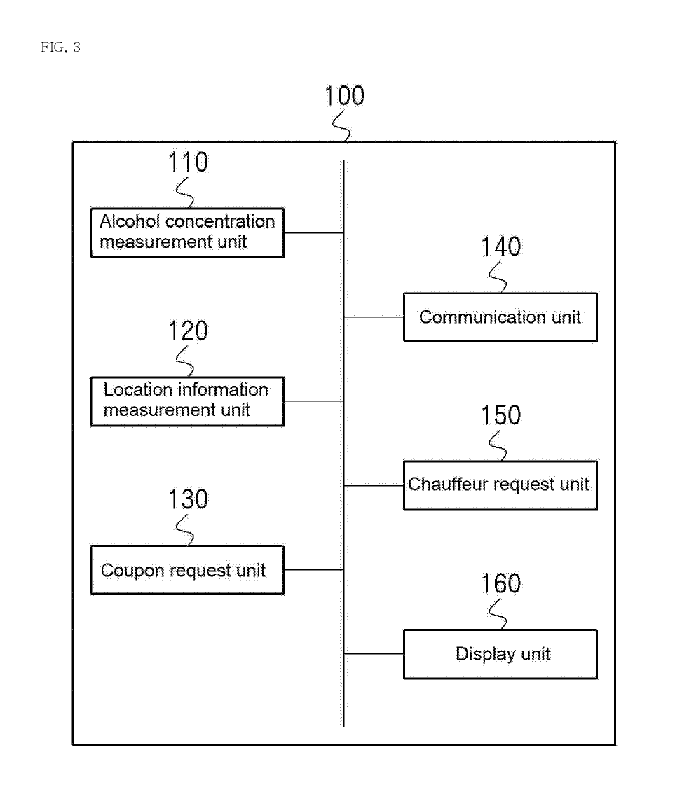

FIG. 3 is a diagram showing the configuration of a function block of the wearable device 100 according to an embodiment of the present invention.

Referring to FIG. 3, the wearable device 100 according to an embodiment of the present invention may include an alcohol concentration measurement unit 110, a location information measurement unit 120, a coupon request unit 130, and a communication unit 140. More specifically, the wearable device 100 may include the alcohol concentration measurement unit 110 configured to measure a blood alcohol level of a user in a predetermined cycle, the location information measurement unit 120 configured to measure information about the location of the user, the coupon request unit 130 configured to request information about a coupon or point which may be used at a store within a predetermined range from the management server 200 when the measured blood alcohol level of the user is a predetermined value or more, and the communication unit 140 configured to transmit and receive information about the blood alcohol level and location of the user to and from an external apparatus over a communication network.

Alcohol included in a breath that is breathed by a person is part of alcohol that has been absorbed by the intestines and entered blood. That is, part of alcohol that has entered blood is mixed with air and exits out of the body. Accordingly, if the amount of alcohol included in the breath is measured, a blood alcohol concentration can be checked. The alcohol concentration measurement unit 110 can measure a blood alcohol level by measuring the amount of alcohol included in a breath that is breathed by a user as described above.

In particular, the alcohol concentration measurement unit 110 may come into contact with any part of the body of a user, and may measure a blood alcohol level of the user in a predetermined cycle by measuring sweat, a body temperature, the heart rate, etc.

In this case, the blood alcohol level of the user can be frequently measured without a user's artificial behavior to directly breathe an outbreath only when the user wears the wearable device 100. Accordingly, overdrinking can be prevented because the blood alcohol level of the user is measured in the predetermined cycle although the user is not conscious of the degree of his or her drinking. In this case, the predetermined cycle also includes the concept of real-time measurement.

When a user drinks, the alcohol concentration measurement unit 110 according to an embodiment of the present invention checks a blood alcohol level of the user in real time although the user does not consciously measure his or her blood alcohol level. Accordingly, the user can refrain from overdrinking and an accident, etc. attributable to excessive drinking can be prevented.

In this case, the alcohol concentration measurement unit 110 may measure the blood alcohol level from sweat discharged from the body of the user.

If a person drinks, an alcoholic ingredient that has entered the body is absorbed by the stomach and the intestines. About 10% of the alcoholic ingredient is not digested and is mixed with respiration, sweat, urine, etc., and discharged to the outside. The remaining 90% is changed into acetic acid while being oxidized in the liver by alcohol catabolic enzyme, supplies energy, decomposed into carbon dioxide, and discharged in the form of respiration. In an embodiment of the present invention, a blood alcohol level of a person can be measured by measuring an alcohol concentration included in discharged sweat.

In addition, the alcohol concentration measurement unit 110 may measure a blood alcohol level based on a change in the body temperature of a user. The alcohol concentration measurement unit 110 may include a sensor capable of measuring a body temperature. In this case, the alcohol concentration measurement unit 110 may sense the temperature of a user prior to drinking, and may measure an alcohol concentration based on the degree that the temperature of the user has risen after drinking.

Furthermore, the alcohol concentration measurement unit 110 may measure a blood alcohol level based on a change in the heart rate of a user. In this case, the alcohol concentration measurement unit 110 may include a pulsation sensor. A sensor for converting a location and a fine pressure signal according to the contraction/relaxation action of the heart through a blood vessel into an electrical signal using the principle of an optical type, a semiconductor type and/or a piezoelectric type and detecting the electrical signal may be used as the pulsation sensor. In this case, the alcohol concentration measurement unit 110 may sense the heart rate of a user prior to drinking, and may measure an alcohol concentration based on the degree that the heart rate of the user has risen after drinking.

For a comparison between the state of a user prior to drinking and the state of the user after drinking, the degree of a change in a body temperature or heart rate before and after drinking may have been sampled. The aforementioned embodiments are only some examples for measuring a blood alcohol level, and a blood alcohol level may be measured in various manners using the wearable device 100.

The alcohol concentration measurement unit 110 may store a blood alcohol level of a user every predetermined time, may check the drinking habit of the user at normal times, and may notify the user of the possibility that a disease may occur due to the drinking habit of the user.

For example, the alcohol concentration measurement unit 110 may record a blood alcohol level of a user daily, and may output the monthly drinking habit of the user in a graph form or table form. If the drinking habit of the user is long, the alcohol concentration measurement unit 110 may provoke the user's attention of drinking by providing a prediction that the user may have a good possibility of having what disease within several years.

In an embodiment of the present invention, in order to record the drinking habit of a user, a storage unit for storing the blood alcohol level of the user measured by the alcohol concentration measurement unit 110 may be used. The storage unit is a device for storing data and may include a main memory device and an auxiliary memory device.

The location information measurement unit 120 may measure information about the location of a user. To this end, a beacon or GPS information may be used.

The beacon is short-range communication means for notifying the management server 200 of information about the location of a user by sending a beacon signal to the location information measurement unit 120 of the wearable device 100 within a short distance (about 50˜70 m). The beacon is a technology in which a beacon installed at a store periodically sends location identification information and a Received Signal Strength Indicator (RSSI) value in a signal form and receives information when the user of the wearable device 100 is within the arrival distance of the beacon signal. The beacon plays the role of a reference point that provides notification of the location. In order to transfer information, a communication technology (Dedicated Short-Range Communications (DSRC), supersonic waves, infrared rays, Bluetooth, CDMA, LTE, Wi-Fi, or Li-Fi) is used. Furthermore, the beacon may be divided into a sound-based low frequency beacon, an LED beacon, a Wi-Fi beacon, a Bluetooth beacon, etc. depending on a transmission method. In particular, in an embodiment of the present invention, short-range wireless communication based on a Bluetooth Low Energy (BLE) protocol may be used.

The BLE beacon is a short-range wireless communication device based on Bluetooth 4.0 protocol, and may communicate with devices within a maximum of 70 m and has high accuracy to the extent that a distinction of a 5˜10 cm unit is possible. Furthermore, the BLE beacon has low power consumption, and is suitable for implementing an Internet of Thing (IoT) in which all of devices are always connected. The beacon can provide a user experience of a space unit because it has a longer available distance than NFC, and can provide indoor location information which is impossible by a GPS technology.

In this case, the location identification information transmitted by the beacon may be the terminal ID number of the beacon or the ID number of a store in which the beacon has been installed. Accordingly, if the wearable device 100 provides the terminal ID number of the beacon to the management server 200, information about an advertisement, a coupon, points, an event, etc. related to the store corresponding to the terminal ID number can be provided to the wearable device 100 because the type and location of the store have been stored in the management server 200.

In an embodiment of the present invention, when a user drinks at a store in which a beacon has been installed, the beacon may send a beacon signal, including location identification information of the store, to the wearable device 100. The wearable device 100 may send the location identification information to the management server 200. The management server 200 may send information about an advertisement related to the store where the user is located or a coupon, points, and event, etc. which may be used in the store. If a beacon has not been installed on the store, the location information measurement unit 120 may send information about the location of the user to the management server 200 using GPS information.

When a blood alcohol level of a user is a predetermined value or more, the coupon request unit 130 may request information about a coupon or point which may be used at a store where the user is located or a store within a predetermined range from the management server 200.

The state of mind and body depending on a blood alcohol level of a person is as follows. If the blood alcohol level is 0.05%, feeling becomes a little better, but there is no great change. If the blood alcohol level is 0.1%, a body response time increases and self-control is weakened. If the blood alcohol level is 0.2%, the sense of sight or the sense of hearing and a language function are reduced. If the blood alcohol level is 0.3%, it is difficult to keep a body balance and the ability to perceive is significantly reduced. If the blood alcohol level is 0.45, a mind becomes confused like dizziness and walking is impossible. If the blood alcohol level is 0.65%, a person may lose his or her consciousness or may fall into a comatose state. Meanwhile, in Korea, driving in the state in which the blood alcohol level is 0.05% or more is classified as drunken driving.

Accordingly, if a blood alcohol level of a user exceeds 0.1%, the coupon request unit 130 may provide a hangover beverage coupon or point which may be used at a store close to the location of the user. If the alcohol concentration is 0.45%, the coupon request unit 130 may provide a food coupon or point for drinking to relieve the handover, which may be used the next day. In addition, if the alcohol concentration is 0.65% or higher, the coupon request unit 130 may provide an accommodations coupon or point or a chauffeur coupon or point because more drinking is dangerous.

The communication unit 140 may enable pieces of information, such as information about a blood alcohol level of a user and information about the location of the user, to be transmitted and received to and from an external apparatus over a communication network. In this case, the communication network includes both wired and wireless communication methods. The wearable device 100 and the management server 200 may be interconnected over the communication network.

The communication unit 140 includes RF transmission means for up-converting a transmitted signal and amplifying the frequency of the signal, RF reception means for performing low-noise amplification on a received signal and down-converting the frequency of the signal, and so on. The communication unit 140 may include at least one of a wireless communication module and a wired communication module. The wireless communication module may include at least one of a wireless network communication module, a Wireless Local Area Network (WLAN) or Wireless Fidelity (Wi-Fi) or Worldwide Interoperability for Microwave Access (WiMAX) communication module, and a Wireless Personal Area Network (WPAN) communication module.

The wireless communication module is an element for transmitting and receiving data in accordance with a wireless communication method. If the wearable device 100 uses wireless communication, it may send or receive data to and from the management server 200 using any one of the wireless network communication module, the WLAN communication module, and the WPAN communication module.

The wireless network communication module functions to access a communication network through a base station and to transmit and receive data. When the wireless network communication module receives information about a blood alcohol level of a user from the alcohol concentration measurement unit 110, it may access a communication network through a base station and send the information about the blood alcohol level to the management server 200. Furthermore, the wireless network communication module may access a communication network through a base station, may receive information about a coupon or point which may be used at a store where a user is located or another store within a predetermined range from the store where the user is located from the management server 200, and may provide the received information to a display unit 160 to be described later.

When the alcohol concentration of the user is a predetermined range or more, the communication unit 140 may send the settlement signal of the user to the management server 200 when the user performs settlement at a food store. Accordingly, the management server 200 may check that the user has performed the settlement at the food store after drinking and may provide a coupon which may be used nearby or send a chauffeur call query, so the user can conveniently request a chauffeur service while preventing drunken driving.

In another embodiment of the present invention, the wearable device 100 may further include a chauffeur request unit 150 configured to query a user whether a chauffeur call request will be transmitted to the management server 200 when a blood alcohol level of the user is a predetermined value or more. In Korea, driving when a blood alcohol level is 0.05% or more is classified as drunken driving. Accordingly, at the moment when a blood alcohol level is 0.05% or more, the wearable device 100 queries a user whether a chauffeur call request will be transmitted to the management server 200. The chauffeur call request may be displayed on the display unit 160. Accordingly, the user can schedule the time by calling a chauffeur service or can notify the management server 200 that he or she will use a chauffeur service later.

In another embodiment of the present invention, the wearable device 100 may further include the display unit 160 configured to display a blood alcohol level of a user in real time and to display a warning message when a blood alcohol level of a user is a predetermined value or more.

The display unit 160 may include a Liquid Crystal Display (LCD), a Thin Film Transistor LCD (TFT-LCD), Organic Light Emitting Diodes (OLED), a Light-Emitting Diode (LED), an Active Matrix Organic LED (AMOLED), a flexible display, a 3-dimensional (3D) display, etc. In this case, the display unit 160 may be formed in a touch screen form. If the display unit 160 is formed in a touch screen form as described above, it may perform all or some of the functions of input means. In particular, the display unit 160 according to an embodiment of the present invention may display a coupon or point received from the management server 200, and may display a blood alcohol level of a user in real time.

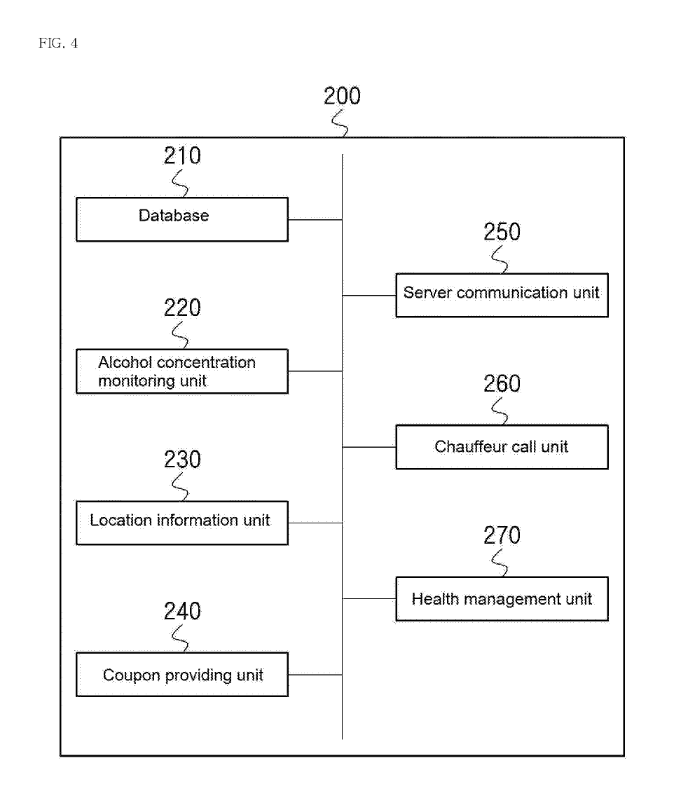

FIG. 4 is a diagram showing the configuration of a function block of the management server 200 according to an embodiment of the present invention.

Referring to FIG. 4, the management server 200 according to an embodiment of the present invention may include a database 210, an alcohol concentration monitoring unit 220, a location information unit 230, a coupon providing unit 240, and a server communication unit 250, 140. More specifically, the management server 200 may include the database 210 configured to store user information and information about a coupon or point which may be used at a stored, the alcohol concentration monitoring unit 220 configured to monitor a real-time blood alcohol level of a user received from the wearable device 100 and to send a warning to the wearable device 100 when the blood alcohol level is a predetermined value or more, the location information unit 230 configured to receive information about the location of the wearable device 100 to check the location of the user of the wearable device 100, the coupon providing unit 240 configured to send a coupon or point which may be used at a store within a predetermined range from the location of the wearable device 100 to the wearable device 100 when a blood alcohol level of a user is a predetermined value or more, and the server communication unit 250, 140 configured to perform the transmission and reception of pieces of information to and from an external apparatus over a communication network.

The database 210 may store user information (e.g., an age, sex, a disease, and the drinking habit of a user), information about a coupon or point which may be used at a store, information about a chauffeur driver (e.g., chauffeur contact information, a chauffeur location, and a chauffeur company) and so on.

The database 210 is a device for storing data and basically stores environment variables, etc. for search, classification, analysis, etc. Such a function of the database 210 may be implemented using a known technology, and thus a detailed description of the implementation is omitted.

The alcohol concentration monitoring unit 220 may monitor a real-time blood alcohol level of a user received from the wearable device 100, and may send a warning to the wearable device 100 if the blood alcohol level is a predetermined value or more.

For example, the alcohol concentration monitoring unit 220 may send a warning message, such as “driving is impossible due to drinking” when a blood alcohol level of a user is 0.05%, “please refrain from exercise because a body response time increases” when the blood alcohol level is 0.1%, “the sense of sight, the sense of hearing and/or a language function is reduced” when the blood alcohol level is 0.2%, “it is difficult to keep a body balance and the ability to perceive is reduced” when the blood alcohol level is 0.3%, “it is a danger level and walking may be difficult” when the blood alcohol level is 0.45, or “you may lose your consciousness if you drink more” when the blood alcohol level is 0.65%, to the wearable device 100.

The location information unit 230 may check the location of the user of the wearable device 100 by receiving information about the location of the wearable device 100. Accordingly, the management server 200 may check the location of a user, may provide a coupon or point based on the location of the user, and may request a chauffeur service from a chauffeur driver close to the location of the user.

When a blood alcohol level of a user is a predetermined value or more, the coupon providing unit 240 may send a coupon or point which may be used at a store within a predetermined range from the location of the wearable device 100 to the wearable device 100. In this case, the coupon providing unit 240 may divide the blood alcohol level of the user into predetermined values and differentially supply the coupon or point.

For example, when a blood alcohol level of a user exceeds 0.1% while the blood alcohol level of the user is monitored, the coupon providing unit 240 may provide a hangover beverage coupon or point which may be used at a store close to the location of the user. When the alcohol concentration is 0.45%, the coupon providing unit 240 may provide a food coupon or point for drinking to relieve the handover, which may be used the next day. Furthermore, when the alcohol concentration is 0.65% or more, the coupon providing unit 240 may provide an accommodations coupon or point or a chauffeur coupon or point because more drinking is dangerous.

The server communication unit 250, 140 may enable the transmission and reception of pieces of information to and from an external apparatus over a communication network. In this case, the communication network includes both wired and wireless communication. The wearable device 100 and the management server 200 may be interconnected over a communication network.

In another embodiment of the present invention, the management server 200 may further include a chauffeur call unit 260 configured to send a chauffeur call query to the wearable device 100 when a blood alcohol level of a user is a predetermined value or more and to send information about the location and destination of the user of the wearable device 100 to the chauffeur driver device 300 within a predetermined range from the location of the wearable device 100 when a chauffeur call request is received from the wearable device 100.

Accordingly, a user who has received a chauffeur call query may determine whether or not to request a chauffeur service through the wearable device 100. If a chauffeur service is not now used, the user may send the time when the chauffeur service is necessary or information about whether the chauffeur service is required or not to the management server 200, so the management server 200 can be prepared to call a chauffeur service. Accordingly, in an embodiment of the present invention, a user can be prevented from drunken driving and can conveniently call a chauffeur service.

In this case, when a settlement signal is received from the wearable device 100, the chauffeur call unit 260 may send a chauffeur call query to the wearable device 100. Accordingly, the management server 200 may check that a user has performed the settlement at a food store after drinking by receiving the settlement signal from the wearable device 100, and may provide a coupon which may be used nearby or send a chauffeur call query to the user so that the user can conveniently call a chauffeur service while the user is prevented from drunken driving.

In another embodiment of the present invention, the management server 200 may further include a health management unit 270 configured to store a blood alcohol level of a user in the database 210, to check the drinking habit of the user, and to notify the user of the possibility that a disease may occur due to the drinking habit of the user.

For example, the health management unit 270 may daily record a blood alcohol level of a user received from the wearable device 100, and may manage the monthly drinking habit of the user in a graph form or table form. If the drinking habit of the user is long, the health management unit 270 may provide a prediction indicating that the possibility that what disease may occur within several years is good in order to give a warning of drinking to the user. In this case, the record of the drinking habit of the user may be stored in the database 210.

The service providing system according to the measurement of a blood alcohol level, the wearable device, and the management server in accordance with embodiments of the present invention are described below from a viewpoint of a method. The steps of a service providing method according to the measurement of a blood alcohol level to be described later have been in connection with the devices according to embodiments of the present invention, and thus a redundant description thereof is omitted.

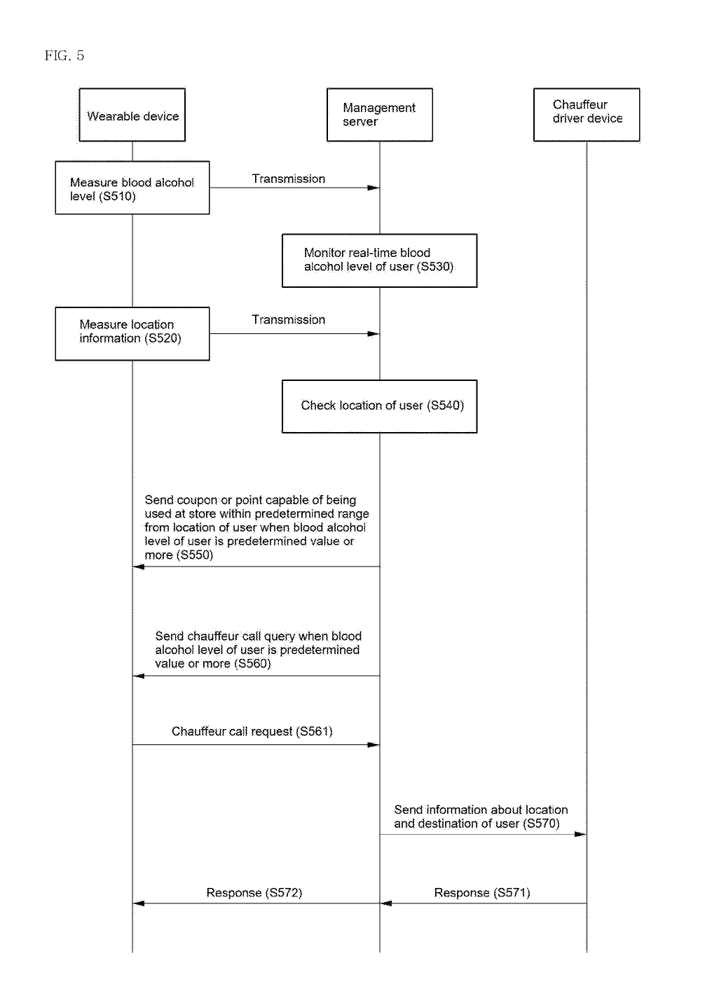

FIG. 5 is a flowchart illustrating a service providing method according to the measurement of a blood alcohol level in accordance with an embodiment of the present invention.

Referring to FIG. 5, the service providing method according to the measurement of a blood alcohol level in accordance with an embodiment of the present invention may include (a) measuring, by the wearable device, a blood alcohol level of a user and sending the measured blood alcohol level to the management server at step S510; (b) measuring, by the wearable device, information about the location of the user and sending the information to the management server at step S520; (c) monitoring, by the management server, a real-time blood alcohol level of the user by receiving information about the real-time blood alcohol level from the wearable device at step S530; (d) checking, by the management server, the location of the user of the wearable device by receiving information about the location of the wearable device at step S540; and (e) sending, by the management server, a coupon or point which may be used at a store within a predetermined range from the location of the wearable device to the wearable device when the blood alcohol level of the user is a predetermined value or more at step S550.

The service providing method may further include (f1) sending, by the management server, a chauffeur call query to the wearable device when the blood alcohol level of the user is a predetermined value or more step S560; and (f2) sending, by the management server, information about the location and destination of the user of the wearable device to a chauffeur driver device within a predetermined range from the location of the wearable device when a chauffeur call request is received from the wearable device at step S570, after (d) step S540.

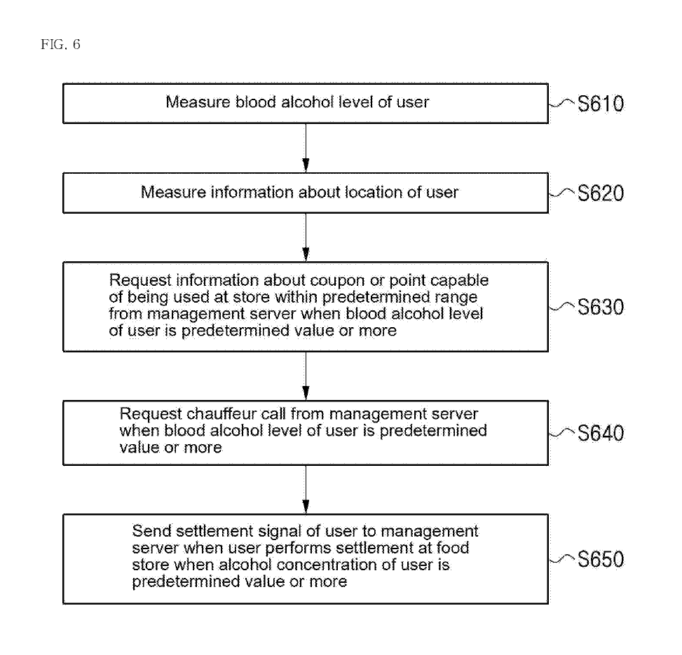

FIG. 6 is a flowchart illustrating a method for providing, by the wearable device, a service according to the measurement of a blood alcohol level in accordance with an embodiment of the present invention.

Referring to FIG. 6, the method for providing, by the wearable device, a service according to the measurement of a blood alcohol level in accordance with an embodiment of the present invention may include (a) measuring a blood alcohol level of a user at step S610; (b) measuring information about the location of the user at step S620; and (c) requesting information about a coupon or point which may be used at a store within a predetermined range from the management server when the blood alcohol level of the user is a predetermined value or more at step S630.

In this case, (a) step S610 may include measuring the alcohol concentration based on at least any one of sweat, a body temperature, and the heart rate of the user.

The method for providing, by the wearable device, a service according to the measurement of a blood alcohol level in accordance with an embodiment of the present invention may further include (d) requesting a chauffeur call from the management server when the blood alcohol level of the user is a predetermined value or more at step S640, after step S620. Step S640 has only to be performed after step S620 and is not limited to a time-series sequence.

Alternatively, the method for providing, by the wearable device, a service according to the measurement of a blood alcohol level in accordance with an embodiment of the present invention may further include (e) sending the settlement signal of the user to the management server when the user performs settlement at a food store when the alcohol concentration of the user is the predetermined value or more at step S650, after (b) step S620). Step S650 has only to be performed after step S620 and is not limited to a time-series sequence.

Furthermore, the method for providing, by the wearable device, a service according to the measurement of a blood alcohol level in accordance with an embodiment of the present invention may further include (f) outputting a blood alcohol level of the user in real time after (b) step S620 and outputting a warning message when the blood alcohol level of the user is the predetermined value or more.

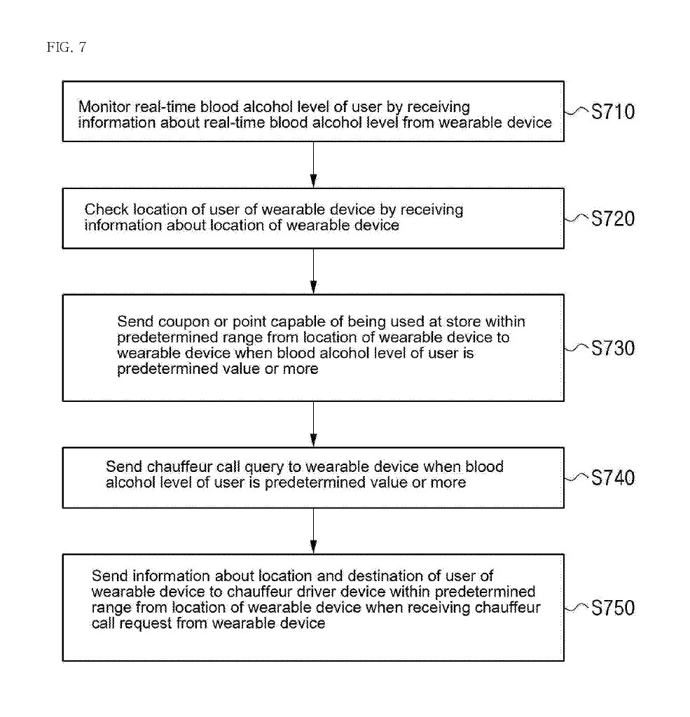

FIG. 7 is a flowchart illustrating a method for providing, by the management server, a service according to the measurement of a blood alcohol level in accordance with an embodiment of the present invention.

Referring to FIG. 7, the method for providing, by the management server, a service according to the measurement of a blood alcohol level in accordance with an embodiment of the present invention may include (a) monitoring a real-time blood alcohol level of a user by receiving information about the real-time blood alcohol level from the wearable device at step S710; (b) checking the location of the user of the wearable device by receiving information about the location of the wearable device at step S720; and (c) sending a coupon or point which may be used at a store within a predetermined range from the location of the wearable device to the wearable device when the blood alcohol level of the user is a predetermined value or more at step S730.

In this case, (c) step S730 may include dividing the blood alcohol level of the user into predetermined values and differentially supplying the coupon or point.

The method for providing, by the management server, a service according to the measurement of a blood alcohol level in accordance with an embodiment of the present invention may further include (d1) sending a chauffeur call query to the wearable device when the blood alcohol level of the user is the predetermined value or more at step S740; and (d2) sending information about the location and destination of the user of the wearable device to the chauffeur driver device within a predetermined range from the location of the wearable device when receiving a chauffeur call request from the wearable device at step S750, after (b) step S720. (d1) and (d2) steps have only to be performed after (b) step S720 and are not limited to a time-series sequence.

Furthermore, (d1) step S740 may include sending a chauffeur call query to the wearable device when a settlement signal is received from the wearable device.

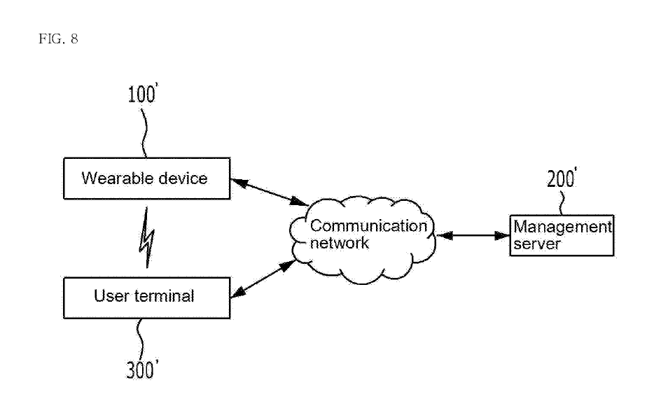

FIG. 8 is a diagram showing the configuration of a function block of a system for measuring a body composition and providing feedback information according to an embodiment of the present invention.

Referring to FIG. 8, the system for measuring a body composition and providing feedback information according to an embodiment of the present invention includes a wearable device 100 and a management server 200. More specifically, the system may include the wearable device 100 configured to measure a body composition or motion of a user, to calculate the quantity of motion based on the measured motion of the user, to send information about the measured body composition or quantity of motion to the management server, and to receive feedback information for the transmitted information from the management server and the management server 200 configured to store and update user information or feedback information, to receive information about the body composition or quantity of motion of the user from the wearable device 100, to select feedback information from the database 210 based on the received information about the body composition or quantity of motion, and to send the selected feedback information to the wearable device 100.

The wearable device 100 literally refers to a device which may be worn on the human body, and includes various types of devices, such as glasses, a watch, a bracelet, shoes, a ring, a belt, a band, a necklace, a headset, and clothing, depending on a part on which the device is worn.

The wearable device 100 used in an embodiment of the present invention may measure a user's body composition. The body composition includes the weight, body water, protein, inorganic matter, body fat, skeletal muscle mass, body mass index (BMI), body fat rate, abdominal obesity rate, diagnosis of obesity, amount of fat control, amount of muscle control, recommended exercise, physical development index, basal metabolic rate, evaluation of body fat for each part, evaluation of muscle development for each part, a body balance, etc. of the human.

In this case, the wearable device 100 includes an electrode, and may use the principle that an electric current well flows into a muscle containing much water and rarely flows into fat less containing water when the electric current flows into the body of a user. Accordingly, a body composition may be measured in such a way as to measure body water, protein, an inorganic matter, and fat, that is, compositions forming the human body, by measuring a resistance value (impedance) which is generated when a fine current passes through the human body. Such a measurement of the body composition is only an example, and the body composition may be measured in various ways using the wearable device 100.

Furthermore, a conventional device for measuring information about exercise of a user using the wearable device 100, etc. has a problem in that the same results are obtained if any user takes exercise and thus has a problem in that an accurate quantity of motion cannot be measured by incorporating information about the body of each user.

In contrast, the wearable device 100 according to an embodiment of the present invention can calculate an accurate quantity of motion of each user by analyzing the body composition and motion of the user, and can receive corresponding feedback from the management server 200 based on the body composition and quantity of motion of the user.

For example, as the results of the analysis of a user's body composition through the wearable device 100, if the user has height of 170 cm, weight of 90 kg, a body fat rate of 30%, an abdominal obesity rate of 10%, and a basal metabolic rate of 2700 kcal, the quantity of 1-day motion including a breast part of 100 kcal and a wrist part of 40 kcal through a user's motion may be measured based on the body composition by measuring the results of the 1-day motion and exercise. Accordingly, when such results of the measurement are transmitted to the management server 200, the management server 200 may send feedback information suitable for the user to the wearable device 100, so the user can obtain various types of information suitable for him or her.

The management server 200 may feed feedback information, more specifically, fitness equipment information, sporting goods information, food information, a coupon information, etc. suitable for a user to the user based on information about the body composition and quantity of motion of the user received from the wearable device 100.

For example, if the body composition of a user received from the wearable device 100 includes height of 170 cm, weight of 90 kg, a body fat rate of 30%, an abdominal obesity rate of 10%, and a basal metabolic rate of 2700 kcal, a received quantity of 1-day motion is a breast part of 100 kcal, and a wrist part 40 of kcal, the management server 200 may provide information about a part that requires more exercise, fitness equipment or an exercise method for the exercise of the part, sporting goods recommended to protect a joint if the user is obesity, food to be eaten and food to be avoided by the user, and stores and a coupon in which the commodities are handled based on information transmitted by the user.

The wearable device 100 or a user terminal 300 to be described later and the management server 200 may be connected over wired/wireless communication networks. The communication network includes a base station controller, a base station transmitter, a relay station, etc. In this case, the base station controller functions to relay a signal between the base station transmitter and a switching station. The communication network supports both a synchronous method and an asynchronous method. Accordingly, in the case of the synchronous method, a Base Station Transmission System (BTS) may become a transmission/reception base station transmitter and a Base Station Controller (BSC) may become a transmission/reception base station controller. In the case of the asynchronous method, a Radio Transceiver Subsystem (RTS) may become the transmission and reception base station transmitter, and a Radio Network Controller (RNC) may become the transmission and reception base station controller. The communication network according to an embodiment of the present invention is not limited thereto, and may collectively refer to a GSM network other than a CDMA network and networks which may be used in the access network of a next-generation mobile communication system to be developed in the future.

The communication network may further include an access point. The access point is a small base station, such as a femto or pico base station chiefly installed in a building. The femto or pico base station is classified depending on how many the wearable devices 100 or the user terminal 300 can be accessed in terms of the classification of a small base station. Furthermore, the access point includes a short-range communication module for performing short-range communication, such as Wi-Fi, along with the wearable device 100 or the user terminal 300. The short-range communication may be performed in accordance with various standards, such as Radio Frequencies (RF) and an ultra-wideband communication (UWB) including Bluetooth communication, Zigbee communication, infrared rays communication (IrDA), a Ultra High Frequency (UHF), and a Very High Frequency (VHF), in addition to Wi-Fi. The access point may extract the location of a data packet, may designate the best communication route for the extracted location, and may transfer the data packet to a next device, for example, the wearable device 100 or the user terminal 300 along the designated communication route. The access point may be shared by several lines in a common network environment.

The access point is basically divided into a fixed type access point and a mobile type access point. The fixed type access point may include a router, a repeater, a relay station, etc. The mobile type access point may include the bridge product of a specific manufacturer, such as KT's Egg. Such a mobile type access point may read a reception-side address from transmission-side information while guaranteeing free mobility, may designate the most appropriate communication route, and may send it.

In another embodiment of the present invention, the system for measuring a body composition and providing feedback information may further include the user terminal 300 configured to store information about a body composition and the quantity of motion measured by the wearable device 100 or feedback information received from the management server and to output the information.

Accordingly, in an embodiment of the present invention, since the wearable device 100 and the management server 200 operate in conjunction with each other even along with the user terminal 300, information about a body composition or the quantity of motion is provided to a user although the user has not worn the wearable device 100. Accordingly, the user can make a plan for his or her exercise while not taking exercise, can receive feedback information from the management server 200, and can easily access several commodities.

The user terminal 300 includes a Personal Digital Assistant (PDA), a smart phone, a cellular phone, a Personal Communication Service (PCS) phone, a Global System for Mobile (GSM) phone, a Wideband CDMA (W-CDMA) phone, a CDMA-2000 phone, a Mobile Broadband System (MSB) phone, etc. which may be applied to various wired/wireless environments. In this case, the user terminal 300 may refer to a portable small-sized device, but may also be called a mobile communication terminal if it includes a camcorder, a laptop computer or the like. In an embodiment of the present invention, the user terminal 300 is not specifically limited thereto.

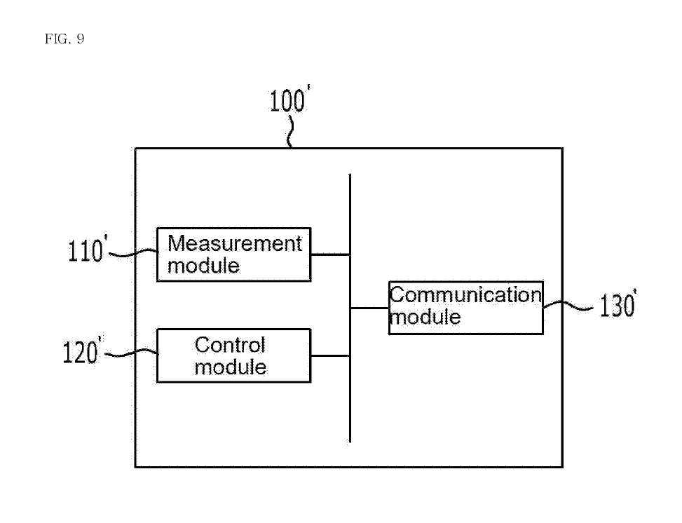

FIG. 9 is a diagram showing the configuration of a function block of the wearable device 100 according to an embodiment of the present invention.

Referring to FIG. 9, the wearable device 100 according to an embodiment of the present invention includes a measurement module 110, a control module 120, and a communication module 130. More specifically, the wearable device 100 may include the measurement module 110 configured to measure a user's body composition or a user's motion, the control module 120 configured to calculate the quantity of motion based on the measured motion of the user, and the communication module 130 configured to send information about the measured body composition or quantity of motion to the management server and to receive feedback information for the transmitted information from the management server.

The measurement module 110 measures a user's body composition or a user's motion. In this case, the body composition includes the weight, body water, protein, inorganic matter, body fat, skeletal muscle mass, body mass index (BMI), body fat rate, abdominal obesity rate, diagnosis of obesity, amount of fat control, amount of muscle control, recommended exercise, physical development index, basal metabolic rate, evaluation of body fat for each part, evaluation of muscle development for each part, a body balance, etc. of the human.

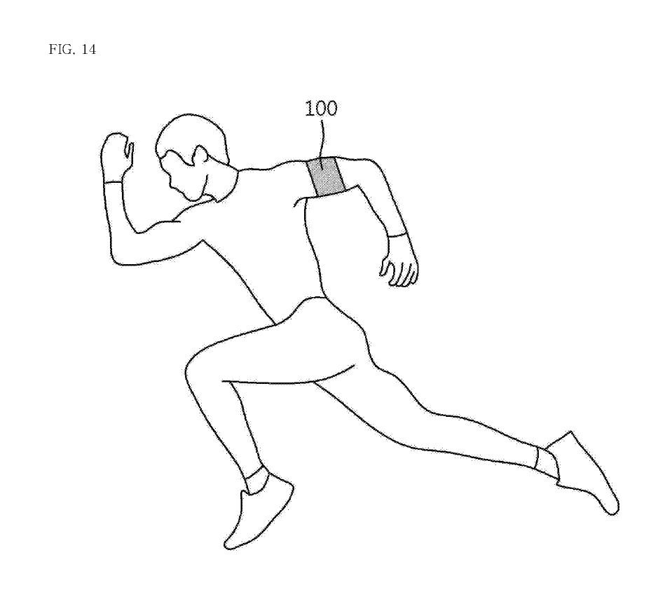

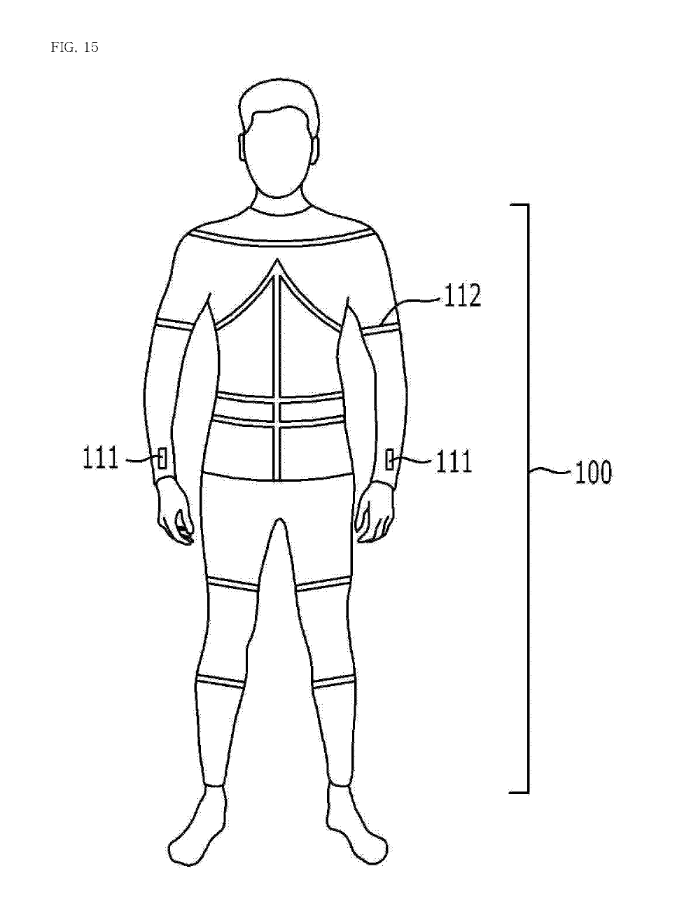

In this case, the measurement module 110 may be attached to any part of the human body as shown in FIG. 14 or may be attached to a plurality of parts as shown in FIG. 15. The measurement module includes an electrode, and may use the principle that an electric current well flows into a muscle containing much water and rarely flows into fat less containing water when the electric current flows into the body of a user. Accordingly, a body composition may be measured in such a way as to measure body water, protein, an inorganic matter, and fat, that is, compositions forming the human body, by measuring a resistance value (impedance) which is generated when a fine current passes through the human body. Such a measurement of the body composition is only an example, and the body composition may be measured in various principles.

Referring to FIG. 15, in another embodiment of the present invention, the measurement module 110 may include a body composition measurement sensor 111 and a motion sensor 112. The body composition measurement sensor 111 may measure information about a body composition, such as the weight, body water, protein, inorganic matter, body fat, skeletal muscle mass, body mass index (BMI), body fat rate, abdominal obesity rate, diagnosis of obesity, amount of fat control, amount of muscle control, recommended exercise, physical development index, basal metabolic rate, evaluation of body fat for each part, evaluation of muscle development for each part, and a body balance of the human, by measuring a resistance value generated when a fine current flows into the body of the human. The motion monitoring sensor may measure motion information, such as a muscle motion, heartbeat, and the respiration volume of a user.

In a conventional method for measuring a body composition, a weight scale type device measures a body composition through the electrodes of feet, but various embodiments according to the location of the measurement module 110 of the wearable device 100 of the present invention are as follows.

<Embodiment According to the Location of the Measurement Module 110>

1) Measurement of Body Composition and Motion Through a Patch Type Wearable Device 100

The body composition measurement sensor 111 of the wearable device 100 attached to part of the body in a patch form may measure a body composition. The motion sensor 112 embedded in the wearable device 100 may measure a motion.

2) Measurement of Body Composition and Motion Through a Breast Belt Type Wearable Device 100

The body composition measurement sensor 111 of the breast belt type wearable device 100 configured to have a form of clothes, such as T-shirts, and attached to surround the breast may measure a body composition. The motion sensor 112 embedded in the wearable device 100 may measure a motion.

3) Measurement of Body Composition and Motion Through a Wrist Type Wearable Device 100

The body composition measurement sensor 111 of the wrist type wearable device 100, such as a smart watch or a smart band, may measure a body composition. The motion sensor 112 embedded in the wearable device 100 may measure a motion.

4) Measurement of Body Composition and Motion Through a Wrist Wearable Device 100 (See FIG. 14)

The body composition measurement sensor 111 of the wrist wearable device 100 having a form capable of being wound on a wrist may measure a body composition. The motion sensor 112 embedded in the wearable device 100 may measure a motion.

5) Measurement of Body Composition and Motion Through a Whole Body Type Wearable Device 100 (See FIG. 15)

The body composition measurement sensor 111 of the wearable device 100 having a clothing form closely attached to the whole body of a user may measure a body composition. The motion sensor 112 embedded in the wearable device 100 may measure a motion.

The aforementioned embodiments of the measurement module are not intended to limit embodiments of the present invention to the proposed forms. Accordingly, those skilled in the art to which the present invention pertains may alter, change, and modify the embodiments without departing from the scope of the present invention.

Furthermore, in another embodiment of the present invention, the measurement module 110 may measure the amount of muscle or body fat of each part of a user and notify the user of an unbalanced part. The measurement module may check the amount of muscle or body fat of each part which belongs to information about the body composition of the human body, may check the amount of activity of each part based on the amount of muscle or body fat, may perform a comparison on the amounts of muscles and body fat of the parts, and may notify the user of an unbalanced part in order to help the user to keep a balanced body.

The control module 120 may calculate the quantity of motion attributable to a motion of a user by incorporating information about a body composition measured by the measurement module 110. In particular, the control module 120 may calculate the quantity of motion by taking into consideration information about at least one of the height, weight, body composition, and sex of the user.

For example, as the results of the analysis of a user's body composition through the measurement module 110, if the user has height of 170 cm, weight of 90 kg, a body fat rate of 30%, an abdominal obesity rate of 10%, and a basal metabolic rate of 2700 kcal, the quantity of 1-day motion including a breast part of 100 kcal and a wrist part of 40 kcal through a user's movement may be measured based on the body composition by measuring the results of the 1-day movement and exercise. Accordingly, when such results of the measurement are transmitted to the management server 200, the management server 200 may send feedback information suitable for the user to the wearable device 100, so the user can obtain various types of information suitable for him or her.

In this case, the feedback information may include at least one of fitness equipment information, sporting goods information, food information, and coupon information corresponding to information about the body of a user. More specifically, the feedback information includes information about a part that requires more exercise, fitness equipment or an exercise method for the exercise of the part, sporting goods recommended to protect a joint if the user is obesity, food to be eaten and food to be avoided by the user, and stores and a coupon in which the commodities are handled based on information transmitted by the user.

The communication module 130 sends information about the measured body composition or quantity of motion to the management server over a communication network, and receives feedback information for the transmitted information from the management server. In this case, the communication network includes both wired and wireless communication, and the wearable device 100 and the management server 200 are interconnected over a communication network.

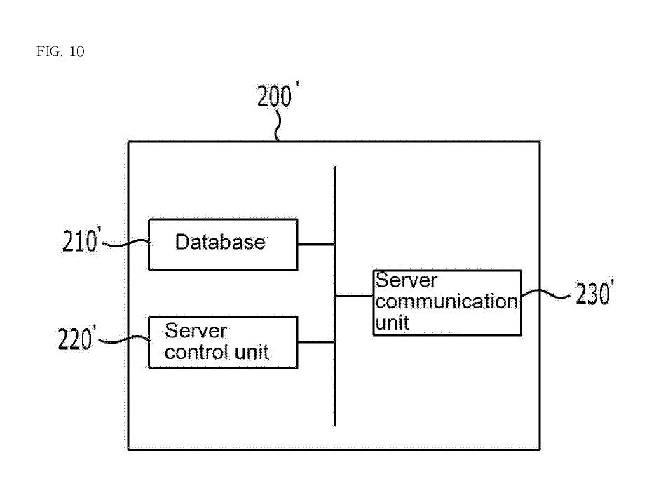

FIG. 10 is a diagram showing the configuration of a function block of the management server 200 according to an embodiment of the present invention.

Referring to FIG. 10, the management server 200 according to an embodiment of the present invention includes a database 210, a server communication unit 230, and a server control unit 220. More specifically, the management server 200 may include the database 210 configured to store and update user information or feedback information, the server communication unit 230 configured to receive information about the body composition or quantity of motion of a user measured by the wearable device 100 and to send information selected by the control unit to the wearable device 100, and the server control unit 220 configured to select feedback information from the database 210 based on the received information about the body composition or quantity of motion.

The database 210 may store information received from the wearable device 100, such as user information (e.g., information about the age, sex, height, weight, and body fat of a user and physical measured values required by the user), fitness equipment information, sporting goods information, food information, and coupon information, and feedback information to be provided to a user, and may update information when new information is received from the wearable device 100 or the external apparatus. In this case, the database 210 is a device for storing data, and basically stores data, such as environment variables for search, classification, and analysis. Such a function of the database 210 may be implemented using a known technology, and thus a detailed description of an implementation is omitted.

The server control unit 220 may select feedback information from the database 210 based on received information about a body composition or the quantity of motion. For example, the database 210 stores different information according to the physical constitution and weight of each person. If a user's body composition received from the wearable device 100 includes height of 170 cm, weight of 90 kg, a body fat rate of 30%, an abdominal obesity rate of 10%, and a basal metabolic rate of 2700 kcal, the server control unit 220 may determine the physical constitution or weight group of the user, and may extract food to be avoided by the user, weight to be reduced for normal weight, a distance that the user needs to walk for a day, calories to be consumed for a day, food to be eaten for a day, food to be avoided by the user if the user is obesity, etc. from the database 210.

In this case, the server control unit 220 may recommend at least one of suitable food, fitness equipment, and sporting goods based on current information about the body of a user so that the user can reach a predetermined target body measurement value. For example, if information about the quantity of motion received from the wearable device 100 includes that the quantity of 1-day motion of a user is a breast part of 100 kcal and a wrist part of 40 kcal, the server control unit 220 may provide information about a part that requires more exercise, fitness equipment or an exercise method for the exercise of the part, sporting goods recommended to protect a joint if the user is obesity, food to be eaten and food to be avoided by the user, and stores and a coupon in which the commodities are handled.

In addition, the server control unit 220 may update information about a user's body composition in real time whenever it receives information about a user's body composition from the wearable device 100. Accordingly, a user can easily check a change of his or her body and required information depending on the quantity of motion daily without a need to write his or her body information separately.

The server communication unit 230 may receive information about the body composition or quantity of motion of a user measured by the wearable device 100 over a communication network, and may send information selected by the server control unit to the wearable device 100. In this case, the communication network includes both wired and wireless communications, and the wearable device 100 and the management server 200 are interconnected over a communication network.

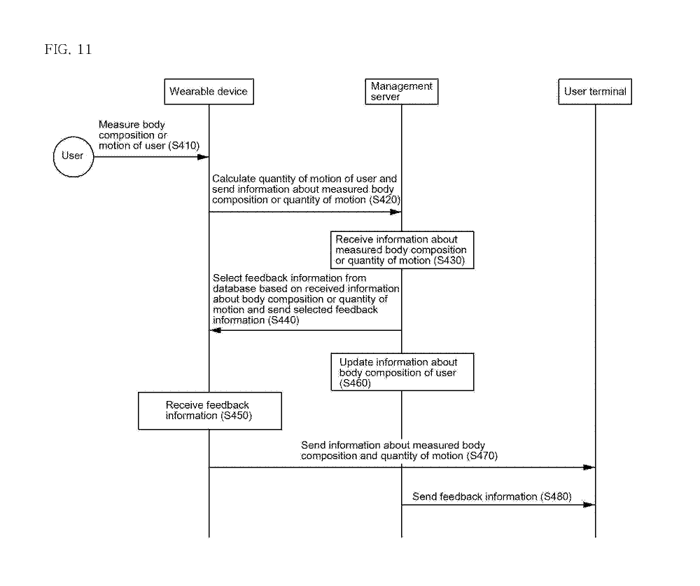

FIG. 11 is a flowchart illustrating a service providing method according to an embodiment of the present invention.

Referring to FIG. 11, the service providing method according to an embodiment of the present invention includes (a) measuring, by the wearable device, the body composition or motion of a user at step S410; (b) calculating, by the wearable device, the quantity of motion based on the measured motion of the user and sending information about the measured body composition or quantity of motion to the management server at step S420; (c) receiving, by the management server, information about the measured body composition or quantity of motion of the user from the wearable device at step S430; (d) selecting, by the management server, feedback information from the database based on the received information about the body composition or quantity of motion and sending the selected feedback information to the wearable device at step S440; and (e) receiving, by the wearable device, the feedback information from the management server at step S450.

The wearable device 100 literally refers to a device which may be worn on the human body, and includes various types of devices, such as glasses, a watch, a bracelet, shoes, a ring, a belt, a band, a necklace, a headset, and clothing, depending on a part on which the device is worn.

At step S410, the wearable device 100 includes an electrode, and may measure a body composition using the principle that an electric current well flows into a muscle containing much water and rarely flows into fat less containing water when the electric current flows into the body of a user. Accordingly, the wearable device 100 may measure a body composition in such a way as to measure body water, protein, an inorganic matter, and fat, that is, compositions forming the human body, by measuring a resistance value (impedance) which is generated when a fine current passes through the human body. Such a measurement of the body composition is only an example, and the body composition may be measured in various ways using the wearable device 100.

In this case, the body composition includes the weight, body water, protein, inorganic matter, body fat, skeletal muscle mass, body mass index (BMI), body fat rate, abdominal obesity rate, diagnosis of obesity, amount of fat control, amount of muscle control, recommended exercise, physical development index, basal metabolic rate, evaluation of body fat for each part, evaluation of muscle development for each part, a body balance, etc. of the human.

Furthermore, a conventional device for measuring information about exercise of a user using the wearable device 100, etc. has a problem in that the same results are obtained if any user takes exercise and thus has a problem in that an accurate quantity of motion cannot be measured by incorporating information about the body of each user.

However, the wearable device 100 according to an embodiment of the present invention can calculate an accurate quantity of motion of each user by analyzing the body composition and motion of the user as at step S420, and can receive corresponding feedback from the management server 200 based on the body composition and quantity of motion of the user.

At step S420, the wearable device 100 calculates the quantity of motion based on the measured motion of the user, and sends information about the measured body composition or quantity of motion to the management server.

For example, as the results of the analysis of a user's body composition through the wearable device 100, if the user has height of 170 cm, weight of 90 kg, a body fat rate of 30%, an abdominal obesity rate of 10%, and a basal metabolic rate of 2700 kcal, the quantity of 1-day motion including a breast part of 100 kcal and a wrist part of 40 kcal through a user's motion may be measured based on the body composition by measuring the results of the 1-day motion and exercise. Accordingly, when such results of the measurement are transmitted to the management server 200, the management server 200 may send feedback information suitable for the user to the wearable device 100, so the user can obtain various types of information suitable for him or her.

At step S430, the management server 200 receives information about the measured body composition or quantity of motion of the user from the wearable device 100. At step S440, the management server 200 selects feedback information from the database 210 based on the received information about the body composition or quantity of motion, and sends the feedback information to the wearable device 100. The management server 200 may feed feedback information suitable for the user, more specifically, fitness equipment information, sporting goods information, food information, coupon information, etc. back to the user based on the information about the body composition and quantity of motion received from the wearable device 100.

For example, if the body composition of a user received from the wearable device 100 includes height of 170 cm, weight of 90 kg, a body fat rate of 30%, an abdominal obesity rate of 10%, and a basal metabolic rate of 2700 kcal, a received quantity of 1-day motion is a breast part of 100 kcal, and a wrist part 40 of kcal, the management server 200 may provide information about a part that requires more exercise, fitness equipment or an exercise method for the exercise of the part, sporting goods recommended to protect a joint if the user is obesity, food to be eaten and food to be avoided by the user, and stores and a coupon in which the commodities are handled.

At step S450, the wearable device 100 receives the feedback information from the management server. The feedback information includes at least any one of user information, fitness equipment information, sporting goods information, food information, and coupon information.

The wearable device 100 or the user terminal 300 and the management server 200 may be connected over a wired or wireless communication network. The communication network includes a base station controller, a base station transmitter, a relay station, etc. In this case, the base station controller functions to relay a signal between the base station transmitter and a switching station. The communication network supports both a synchronous method and an asynchronous method. Accordingly, in the case of the synchronous method, a Base Station Transmission System (BTS) may become a transmission/reception base station transmitter and a Base Station Controller (BSC) may become a transmission/reception base station controller. In the case of the asynchronous method, a Radio Transceiver Subsystem (RTS) may become the transmission and reception base station transmitter, and a Radio Network Controller (RNC) may become the transmission and reception base station controller. The communication network according to an embodiment of the present invention is not limited thereto, and may collectively refer to a GSM network other than a CDMA network and networks which may be used in the access network of a next-generation mobile communication system to be developed in the future.

The communication network may further include an access point. The access point is a small base station, such as a femto or pico base station chiefly installed in a building. The femto or pico base station is classified depending on how many the wearable devices 100 or the user terminal 300 can be accessed in terms of the classification of a small base station. Furthermore, the access point includes a short-range communication module for performing short-range communication, such as Wi-Fi, along with the wearable device 100 or the user terminal 300. The short-range communication may be performed in accordance with various standards, such as Radio Frequencies (RF) and an ultra-wideband communication (UWB) including Bluetooth communication, Zigbee communication, infrared rays communication (IrDA), a Ultra High Frequency (UHF), and a Very High Frequency (VHF), in addition to Wi-Fi. The access point may extract the location of a data packet, may designate the best communication route for the extracted location, and may transfer the data packet to a next device, for example, the wearable device 100 or the user terminal 300 along the designated communication route. The access point may be shared by several lines in a common network environment.

The access point is basically divided into a fixed type access point and a mobile type access point. The fixed type access point may include a router, a repeater, a relay station, etc. The mobile type access point may include the bridge product of a specific manufacturer, such as KT's Egg. Such a mobile type access point may read a reception-side address from transmission-side information while guaranteeing free mobility, may designate the most appropriate communication route, and may send it.

In another embodiment of the present invention, the system for measuring a body composition and providing feedback information may further include the user terminal 300 configured to store information about a body composition and the quantity of motion measured by the wearable device 100 or feedback information received from the management server and to output the information.

In another embodiment of the present invention, the service providing method may further include (f) updating, by the management server, information about the body composition of each user in real time whenever it receives information about the body composition of the user from the wearable device 100 at step S460. In this case, (f) step is possible at any time without being limited to a time-series sequence.

The service providing method may further include may further include (g) sending, by the wearable device 100, information about the measured body composition and quantity of motion to the user terminal 300 at step S470 and (h) sending, by the management server, the feedback information to the user terminal 300 at step S480 so that even the user terminal 300 operates in conjunction with the wearable device 100 and the management server. Accordingly, a user can make a plan for his or her exercise while not taking exercise, can receive feedback information from the management server 200, and can easily access several commodities because information about a body composition or quantity of motion is provided to the user while the user does not wear the wearable device 100.

The user terminal 300 includes a Personal Digital Assistant (PDA), a smart phone, a cellular phone, a Personal Communication Service (PCS) phone, a Global System for Mobile (GSM) phone, a Wideband CDMA (W-CDMA) phone, a CDMA-2000 phone, a Mobile Broadband System (MSB) phone, etc. which may be applied to various wired/wireless environments. In this case, the user terminal 300 may refer to a portable small-sized device, but may also be called a mobile communication terminal if it includes a camcorder, a laptop computer or the like. In an embodiment of the present invention, the user terminal 300 is not specifically limited thereto.

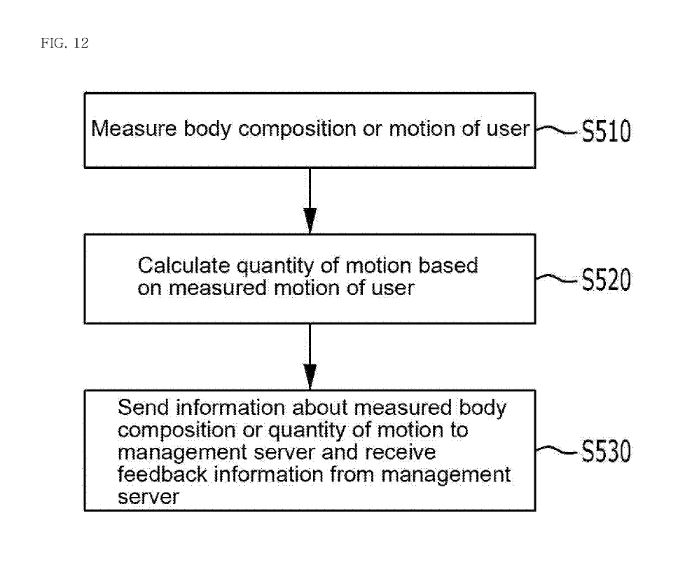

FIG. 12 is a diagram showing a flowchart of a service providing method in a method for measuring, by the wearable device 100, a body composition and providing feedback information according to an embodiment of the present invention.

Referring to FIG. 12, in the method for measuring, by the wearable device 100, a body composition and providing feedback information according to an embodiment of the present invention, the service providing method includes (a) measuring, by the measurement module, the body composition or motion of a user at step S510; (b) calculating, by the control module, the quantity of motion based on the measured motion of the user at step S520; and (c) sending, by the communication module, information about the measured body composition or quantity of motion to the management server and receiving feedback information for the transmitted information from the management server at step S530.

At step S510, the measurement module 110 measures the body composition or motion of the user. In this case, the body composition includes the weight, body water, protein, inorganic matter, body fat, skeletal muscle mass, body mass index (BMI), body fat rate, abdominal obesity rate, diagnosis of obesity, amount of fat control, amount of muscle control, recommended exercise, physical development index, basal metabolic rate, evaluation of body fat for each part, evaluation of muscle development for each part, a body balance, etc. of the human.

In this case, the measurement module 110 may be attached to any part of the human body or may be attached to a plurality of parts. The measurement module includes an electrode, and may use the principle that an electric current well flows into a muscle containing much water and rarely flows into fat less containing water when the electric current flows into the body of a user. Accordingly, a body composition may be measured in such a way as to measure body water, protein, an inorganic matter, and fat, that is, compositions forming the human body, by measuring a resistance value (impedance) which is generated when a fine current passes through the human body. Such a measurement of the body composition is only an example, and the body composition may be measured in various principles.

Meanwhile, (a) step S510 may include measuring, by the body composition measurement sensor 111, a body composition by measuring a resistance value generated when an electric current flows into the body of the user; and measuring, by the motion sensor 112, at least any one of the muscle motion, heartbeat, and respiration volume of the user. The body composition measurement sensor 111 may measure information about a body composition, such as the weight, body water, protein, inorganic matter, body fat, skeletal muscle mass, body mass index (BMI), body fat rate, abdominal obesity rate, diagnosis of obesity, amount of fat control, amount of muscle control, recommended exercise, physical development index, basal metabolic rate, evaluation of body fat for each part, evaluation of muscle development for each part, and a body balance of the human, by measuring a resistance value generated when a fine current flows into the body of the use. The motion monitoring sensor may measure motion information, such as a muscle motion, heartbeat, and the respiration volume of a user.

In a conventional method for measuring a body composition, a weight scale type device measures a body composition through the electrodes of feet, but various embodiments according to the location of the measurement module 110 of the wearable device 100 of the present invention are as follows.

<Embodiment According to the Location of the Measurement Module 110>

1) Measurement of Body Composition and Motion Through a Patch Type Wearable Device 100

The body composition measurement sensor 111 of the wearable device 100 attached to part of the body in a patch form may measure a body composition. The motion sensor 112 embedded in the wearable device 100 may measure a motion.

2) Measurement of Body Composition and Motion Through a Breast Belt Type Wearable Device 100

The body composition measurement sensor 111 of the breast belt type wearable device 100 configured to have a form of clothes, such as T-shirts, and attached to surround the breast may measure a body composition. The motion sensor 112 embedded in the wearable device 100 may measure a motion.

3) Measurement of Body Composition and Motion Through a Wrist Type Wearable Device 100

The body composition measurement sensor 111 of the wrist type wearable device 100, such as a smart watch or a smart band, may measure a body composition. The motion sensor 112 embedded in the wearable device 100 may measure a motion.

4) Measurement of Body Composition and Motion Through a Wrist Wearable Device 100 (See FIG. 14)

The body composition measurement sensor 111 of the wrist wearable device 100 having a form capable of being wound on a wrist may measure a body composition. The motion sensor 112 embedded in the wearable device 100 may measure a motion.

5) Measurement of Body Composition and Motion Through a Whole Body Type Wearable Device 100 (See FIG. 15)

The body composition measurement sensor 111 of the wearable device 100 having a clothing form closely attached to the whole body of a user may measure a body composition. The motion sensor 112 embedded in the wearable device 100 may measure a motion. The aforementioned embodiments of the measurement module are not intended to limit embodiments of the present invention to the proposed forms. Accordingly, those skilled in the art to which the present invention pertains may alter, change, and modify the embodiments without departing from the scope of the present invention.

In addition, at step S510, the measurement module 110 may measure the amount of muscle or body fat of each part of the user and notify an unbalanced part of the user. The measurement module may check the amount of muscle or body fat of each part which belongs to information about the body composition of the human body, may check the amount of activity of each part based on the amount of muscle or body fat, may perform a comparison on the amounts of muscles and body fat of the parts, and may notify the user of an unbalanced part in order to help the user to keep a balanced body.

At step S520, the control module 120 may calculate the quantity of motion attributable to a motion of a user by incorporating information about a body composition measured by the measurement module 110. In particular, at step S520, the quantity of motion may be calculated by taking into consideration information about at least one of the height, weight, body composition, and sex of the user.

For example, as the results of the analysis of a user's body composition through the measurement module 110, if the user has height of 170 cm, weight of 90 kg, a body fat rate of 30%, an abdominal obesity rate of 10%, and a basal metabolic rate of 2700 kcal, the quantity of 1-day motion including a breast part of 100 kcal and a wrist part of 40 kcal through a user's movement may be measured based on the body composition by measuring the results of the 1-day movement and exercise. Accordingly, when such results of the measurement are transmitted to the management server 200, the management server 200 may send feedback information suitable for the user to the wearable device 100, so the user can obtain various types of information suitable for him or her.

In this case, the feedback information may include at least one of fitness equipment information, sporting goods information, food information, and coupon information corresponding to information about the body of a user. More specifically, the feedback information includes information about a part that requires more exercise, fitness equipment or an exercise method for the exercise of the part, sporting goods recommended to protect a joint if the user is obesity, food to be eaten and food to be avoided by the user, and stores and a coupon in which the commodities are handled based on information transmitted by the user.

At step S530, the communication module 130 sends information about the measured body composition or quantity of motion to the management server over a communication network, and receives feedback information for the transmitted information from the management server. In this case, the communication network includes both wired and wireless communication, and the wearable device 100 and the management server 200 are interconnected over a communication network.

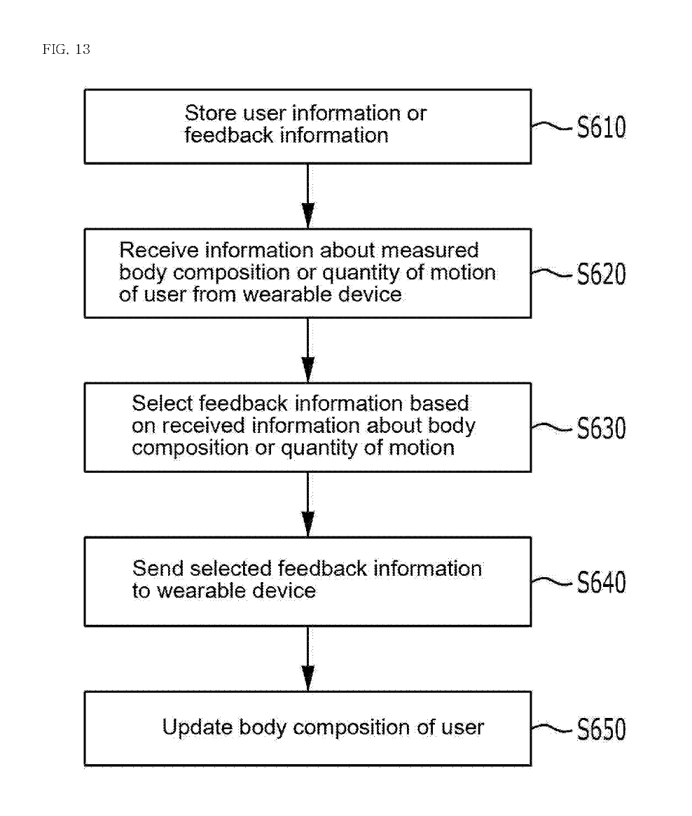

FIG. 13 is a diagram showing a flowchart of a service providing method in a method for measuring, by the management server 200, a body composition and providing feedback information according to an embodiment of the present invention.

Referring to FIG. 13, the method for measuring, by the management server 200, a body composition and providing feedback information according to an embodiment of the present invention includes (a) storing, by the database, user information or feedback information at step S610; (b) receiving, by the server communication unit, information about the measured body composition or quantity of motion of a user from the wearable device at step S620; (c) selecting, by the server control unit, feedback information from the database based on the received information about the body composition or quantity of motion at step S630; and (d) sending, by the server communication unit, the feedback information selected by the server control unit, to the wearable device at step S640.

At step S610, the database 210 stores information received from the wearable device 100, such as user information (e.g., information about the age, sex, height, weight, and body fat of a user and physical measured values required by the user), fitness equipment information, sporting goods information, food information, and coupon information, and feedback information to be provided to a user. In this case, the database 210 basically stores data, such as environment variables for search, classification, and analysis. Such a function of the database 210 may be implemented using a known technology, and thus a detailed description of an implementation is omitted.

At step S620, information about the measured body composition or quantity of motion of the user is received from the wearable device 100 over a communication network. Thereafter, at step S630, the server control unit 220 selects feedback information from the database 210 based on received information about a body composition or the quantity of motion. For example, the database 210 stores different information according to the physical constitution and weight of each person. If a user's body composition received from the wearable device 100 includes height of 170 cm, weight of 90 kg, a body fat rate of 30%, an abdominal obesity rate of 10%, and a basal metabolic rate of 2700 kcal, the server control unit 220 may determine the physical constitution or weight group of the user, and may extract food to be avoided by the user, weight to be reduced for normal weight, a distance that the user needs to walk for a day, calories to be consumed for a day, food to be eaten for a day, food to be avoided by the user if the user is obesity, etc. from the database 210.

In this case, at step S630, at least one of suitable food, fitness equipment, and sporting goods may be recommended based on current information about the body of a user so that the user can reach a predetermined target body measurement value. For example, if information about the quantity of motion received from the wearable device 100 includes that the quantity of 1-day motion of a user is a breast part of 100 kcal and a wrist part of 40 kcal, the server control unit 220 may provide information about a part that requires more exercise, fitness equipment or an exercise method for the exercise of the part, sporting goods recommended to protect a joint if the user is obesity, food to be eaten and food to be avoided by the user, and stores and a coupon in which the commodities are handled.

At step S640, the server communication unit 230 sends the information, selected by the server control unit 220, to the wearable device 100. In this case, the communication network includes both wired and wireless communication, and the wearable device 100 and the management server 200 are interconnected over a communication network.

Furthermore, in an embodiment of the present invention, the method for measuring, by the management server 200, a body composition and providing feedback information may further include (e) updating information about the body composition of the user in real time whenever information about the body composition of the user is received from the wearable device 100 at step S650. Accordingly, a user can easily check a change of his or her body and required information depending on the quantity of motion daily without a need to write his or her body information separately. In this case, (e) step may be possible at any time without being limited to a time-series sequence.

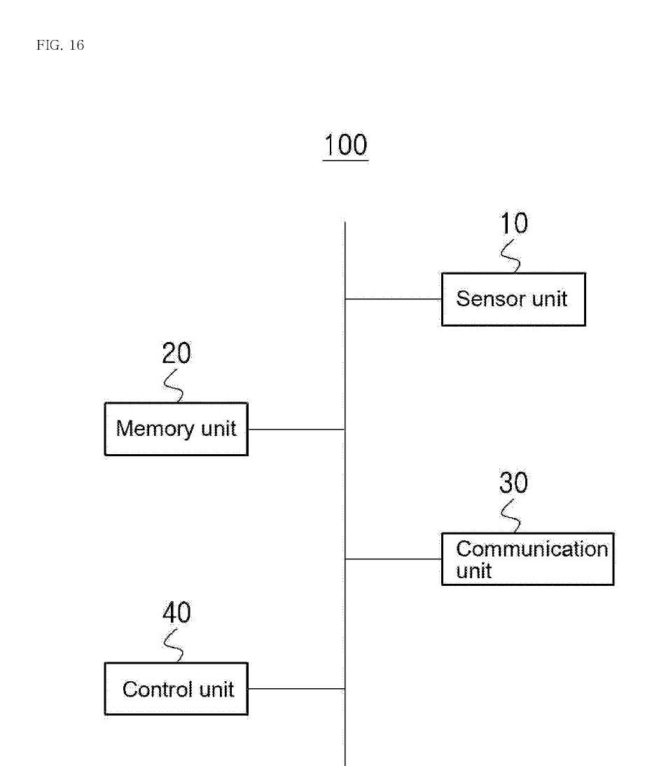

FIG. 16 is a diagram showing major elements of the wearable device 100. The wearable device 100 includes a sensor unit 10, a memory unit 20, a communication unit 30, and a control unit 40. However, the major elements of the wearable device 100 correspond to only one embodiment, but some of the major elements may be omitted or some elements may be added to the major elements, if necessary.

The sensor unit 10 is mounted on the wearable device 100 and measures various types of information from a user. In this case, the type of sensor included in the sensor unit 10 is not limited, and information to be measured is not limited. For example, the sensor unit 10 may include one or more of a behavior recognition sensor, a camera sensor, an infrared sensor, a GPS sensor, a Gyro sensor, a displacement sensor, a pressure sensor, a torque sensor, a proximity sensor, a gravity sensor, an image sensor, a sweat sensor, and other known sensors. However, the sensor unit 10 of the wearable device 100 for measuring a composition ratio of sweat secreted from a user may include a sweat sensor capable of measuring a composition ratio of sweat because each of the sensors can measure determined information.

The memory unit 20 stores various types of information measured by the sensor unit 10 from a user, more specifically, a composition ratio of sweat secreted from a user as first information. In this case, known storage media may be used as the memory unit 20. For example, one or more of known storage media, such as ROM, PROM, EPROM, EEPROM, and RAM, may be used as the memory unit 20. The memory unit 20 may continue to store new information, measured by the sensor unit 10, in the wearable device 100. In this case, a storage medium capable of inputting and deleting information may be used as the memory unit 20 because existing information needs to be deleted depending on the capacity of the memory unit 20.

The communication unit 30 sends first information stored in the memory unit 20 to an information providing apparatus, and receives second information generated by the information providing apparatus by analyzing the first information. That is, the communication unit 30 functions to transmit and receive information between the wearable device 100 and the information providing apparatus. The communication unit 30 may use any known communication methods, such as CDMA, WCDMA, GSM, 3G, and 4G. Accordingly, communication means mounted on a mobile communication terminal, such as a smart phone, may be used as the communication unit 30 without any change.

The control unit 40 outputs second information, received by the communication unit 30 from the information providing apparatus, to any one of a display unit 42 and a speaker unit. Accordingly, the control unit 40 may control the driving of the display unit 42 and the speaker unit, and may also control the driving of the sensor unit 10, the memory unit 20, and the communication unit 30. That is, the control unit 40 may be considered to be a kind of central processing unit (CPU) for controlling overall driving of the wearable device 100.

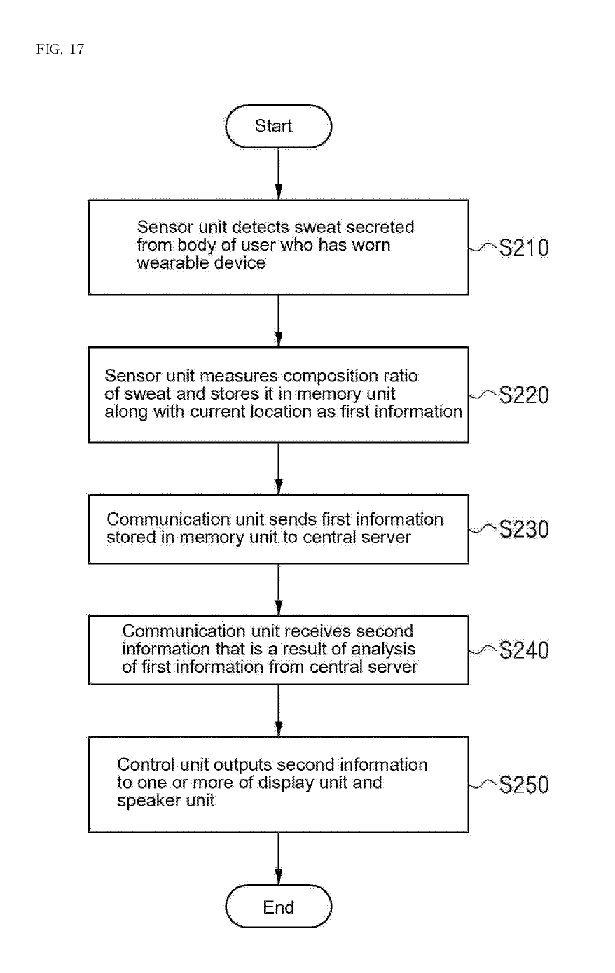

A service providing method according to a first embodiment of the present invention may be performed more effectively through the sensor unit 10, the memory unit 20, the communication unit 30, and the control unit 40. The service providing method is described below with reference to FIG. 17.

FIG. 17 is a flowchart illustrating the service providing method according to the first embodiment of the present invention.

The service providing method is only one embodiment including exemplary steps according to an embodiment of the present invention, and some of the steps may be modified or deleted or some other steps may be added to the exemplary steps.

First, the sensor unit 10 detects sweat secreted from the body of a user who has worn the wearable device 100 at step S210. In this case, the wearable device 100 may include various types of wearable devices. More specifically, the wearable device 100 may include any one of a smart watch, smart glasses, and a smart band. An example in which the wearable device 100 is a smart watch is described below.

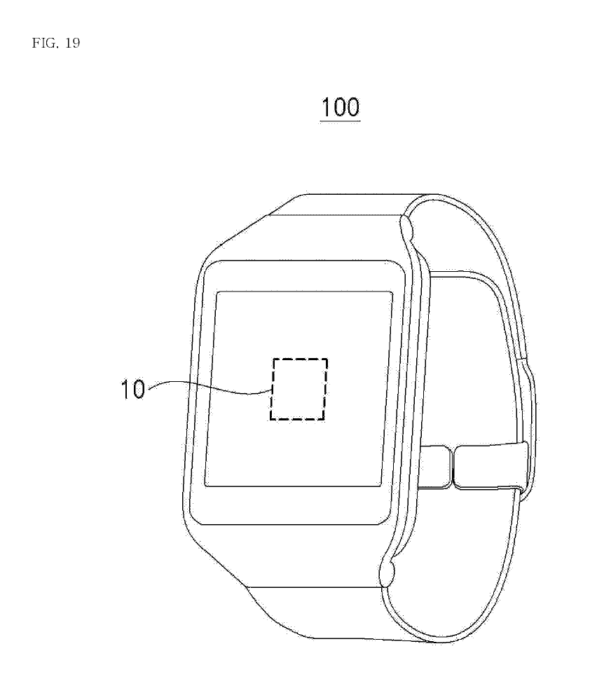

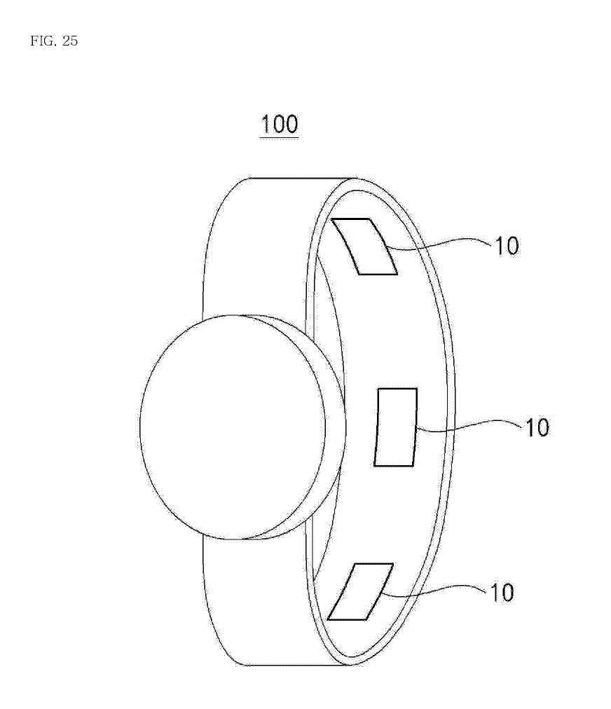

A user may have a smart watch functioning as the wearable device 100 worn on his or her wrist. The sensor unit 10 may be mounted on a portion coming into contact with the wrist of the user. For example, the sensor unit 10 may be mounted on the inside of the strap of the smart watch as shown in FIG. 25 or may be mounted on the back of the main body of the smart watch as shown in FIG. 19. In this case, there is an advantage in that the sensor unit 10 can directly detect sweat secreted from the wrist of a user. In some embodiments, the sensor unit 10 may be mounted on any proper location where sweat secreted from the wrist of the user may be directly detected in addition to the inside of the strap of the smart watch and the back of the main body of the smart watch. Furthermore, one or more sensor units 10 may be used. For example, the sensor units 10 may be formed in the inside of the strap of the smart watch and the back of the main body of the smart watch. In this case, there is an advantage in that a composition ratio of a user can be measured by more accurately detecting sweat which may be differentially secreted depending on a specific part of the wrist of the user.

When the sensor unit 10 detects the sweat secreted from the body of the user, it measures a composition ratio of the detected sweat and stores the measured composition ratio in the memory unit 20 as first information along with the current location of the user at step S220. In this case, the sensor unit 10 may include a sweat sensor of various sensors because it needs to measure the composition ratio of the sweat. The sweat sensor is a contact type sensor, and is capable of detecting sweat and analyzing a composition ratio. The sweat sensor is capable of a composition ratio of compositions forming the sweat secreted from the user. The sweat sensor is capable of detecting sweat secreted from a user, and of measuring a composition ratio, such as water 99%, sodium 0.4%, chlorine 0.1%, potassium 0.1%, a nitrogen-containing component 0.2%, lactic acid 0.1%, and urea 0.1%. In this case, the composition ratio of sweat is only one embodiment. The composition ratio may be different depending on the current state of the body of a user, and some compositions may have a composition ratio of 0.

The sensor unit 10 stores the composition ratio of the sweat of the user in the memory unit 20 as the first information along with the current location of the user. In this case, the current location of the user may be obtained through various means, and may be obtained through a global positioning system (GPS) module, for example. If the current location of the user has been obtained through the GPS module, the first information may include the composition ratio of the sweat of the user, such as water 99%, sodium 0.4%, chlorine 0.1%, potassium 0.1%, a nitrogen-containing component 0.2%, lactic acid 0.1%, and urea 0.1%, and the current location of the user, such as (43.943°, 4.805°). If the current location of the user is obtained using another means other than the GPS module, it may be included in first information according to the corresponding means.

The number of pieces of the first information stored in the memory unit 20 by the sensor unit 10 may be one or more. For example, after first information including a composition ratio of sweat of a user, such as water 99%, sodium 0.4%, chlorine 0.1%, potassium 0.1%, a nitrogen-containing component 0.2%, lactic acid 0.1%, and urea 0.1%, and the current location of the user, such as (43.943°, 4.805°), is stored, new first information including a composition ratio of sweat of the user, such as water 99%, sodium 0.4%, chlorine 0.2%, potassium 0.1%, a nitrogen-containing component 0.1%, and lactic acid 0.2%, and the current location of the user, such as (22.452°, 7.245°), may be additionally stored. In this case, the existing first information may be deleted, or may be accumulated without being deleted depending on the capacity of the memory unit 20 and stored as new first information. Furthermore, the memory unit 20 may delete existing first information when new first information is stored from the time when first information having a specific amount or more is stored because the capacity of the memory unit 20 may be exceeded if the first information is accumulated and stored. That is, various storage methods may be used by the memory unit 20.

When the first information is stored in the memory unit 20, the communication unit 30 sends the first information, stored in the memory unit 20, to the information providing apparatus at step S230. In this case, the information providing apparatus is the management server 200 according to a second embodiment of the present invention, and will be described in detail later. The communication unit 30 may send the first information using any one of the known communication methods, such as CDMA, WCDMA, GSM, 3G, and 4G, as described above.

When the communication unit 30 sends the first information to the information providing apparatus, the information providing apparatus generates second information by analyzing the first information. The communication unit 30 receives the second information from the information providing apparatus at step S240. The control unit 40 may enable the received second information to be stored in the memory unit 20 at step S241. In this case, the second information may be merged with the location corresponding to the first information, that is, the basis for generating information, or the first information. For example, if the first information has been stored in a location A of the memory unit 20, the second information may be stored in a location A′ of the memory unit 20 or may be stored along with the location A. Furthermore, existing first information may be deleted depending on the capacity of the memory unit 20, and second information may be stored in the location from which the existing first information has been deleted. For example, if first information has been stored in a location A of the memory unit 20, the control unit 40 may delete the first information from the location A by detecting that the communication unit 30 has received second information, and may newly store the second information in the location from which the first information has been deleted. The second information includes information about a commodity to be recommended to the user in connection with the composition ratio of the sweat of the user included in the first information. The second information may include information about the closest place where a commodity to be recommended to the user is handled from the current location of the user. This is described in detail later in connection with the management server 200 according to a second embodiment of the present invention.

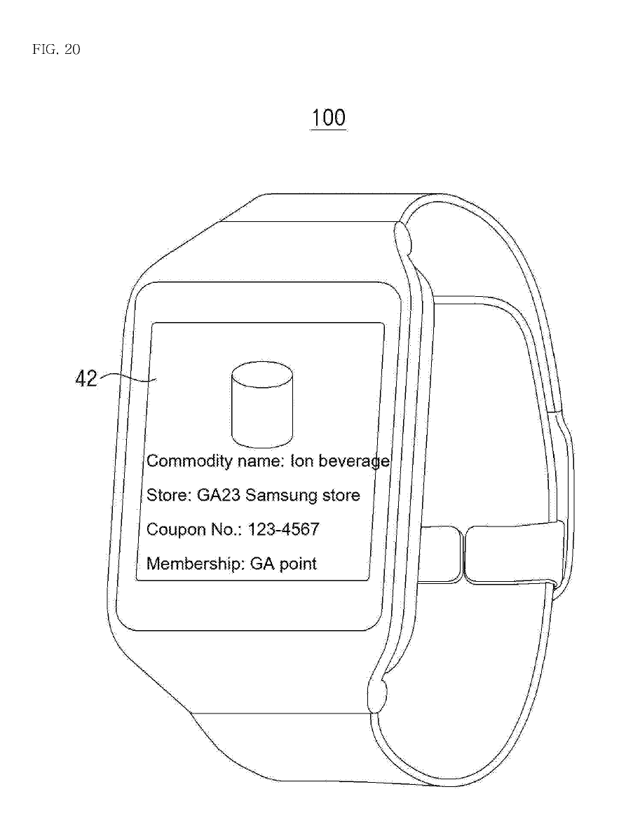

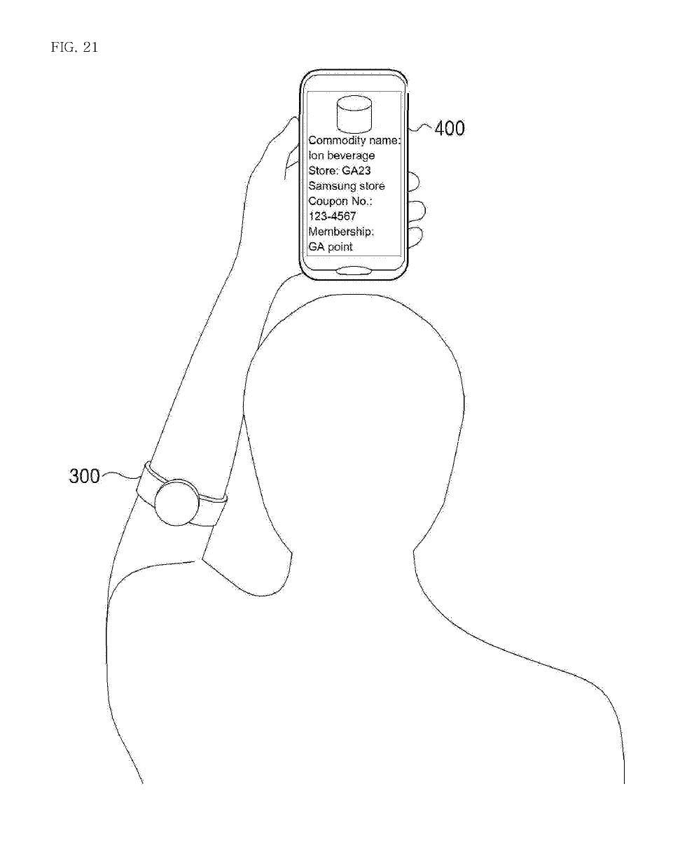

When the communication unit 30 receives the second information, the control unit 40 outputs the second information to one or more of the display unit 42 and the speaker unit at step S250. From FIG. 20, the state in which the second information has been output through a smart watch can be seen. The second information may be output to the display unit 42 and the speaker unit at the same time. In this case, the wearable device 100 may include both the display unit 42 and the speaker unit. If the wearable device 100 is a smart watch, there is no problem because the wearable device 100 includes both the display unit 42 and the speaker unit. In the case of a wearable device 100 not including both the display unit 42 and the speaker unit, for example, a smart band, etc., there may be a problem in outputting the second information. In this case, the control unit 40 may output the second information through another device capable of being paired with a smart band through specific means, such as Bluetooth. In this case, the type of another device is not limited, and may include any device having visual or auditory output means, such as an earphone, a smart phone, and smart glasses which support a Bluetooth function. From FIG. 21, the state in which the second information is output through a smart phone 400 owned by a user who has worn a smart band 300 can be seen.

A composition ratio of sweat of a user can be analyzed and useful information according to the current state of the body of the user can be provided through a smart watch through steps S210 to S250. The smart watch is one of representative wearable devices which are worn on the wrist of a user and is worn on the location where sweat secreted from the user may be directly detected. Accordingly, the smart watch corresponds to an optimum wearable device 100 in implementing a service providing method according to an embodiment of the present invention. In another embodiment, an example in which the wearable device 100 is smart glasses is described below.

First, the sensor unit 10 detects sweat secreted from the body of a user who has worn the wearable device 100 at step S210′. In this case, the wearable device 100 may include various types of wearable devices. More specifically, the wearable device 100 may include any one of a smart watch, smart glasses, and a smart band. In the following example, the wearable device 100 is assumed to be smart glasses.

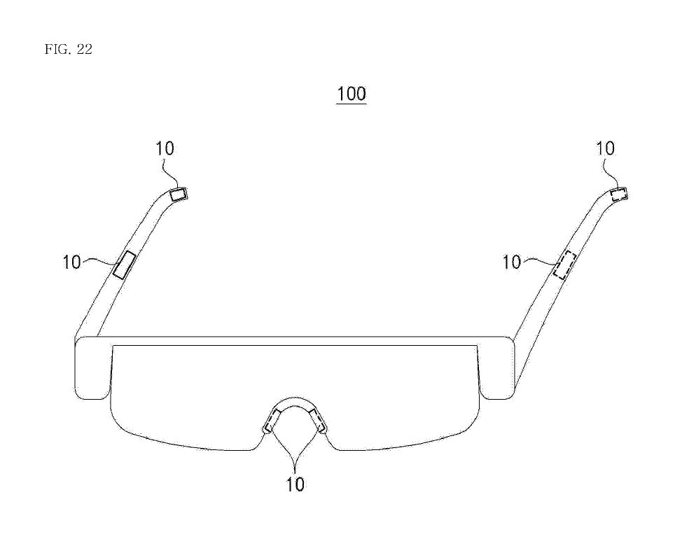

A user wears smart glasses functioning as the wearable device 100. The sensor unit 10 may be mounted on a portion coming into contact with the skin of the user. For example, the sensor unit 10 may be mounted on one or more of the inside of the nose pad and inside of the end piece of the smart glasses. In this case, there is an advantage in that the sensor unit 10 can directly detect sweat secreted from a face of the user. In some embodiments, the sensor unit 10 may be mounted on any proper location from which sweat secreted from a face of the user can be directly detected in addition to the nose pad, inside of the temple, and inside of the end piece of the smart glasses. Furthermore, one or more sensor units 10 may be used. For example, the sensor units 10 may be formed in the nose pad, inside of the temple, and insides of the end pieces of the smart glasses at the same time as shown in FIG. 22. In this case, there is an advantage in that a composition ratio can be measured by more accurately detecting sweat which may be differently secreted depending on a specific part of a face of a user.

When the sensor unit 10 detects the sweat secreted from the body of the user, it measures a composition ratio of the detected sweat and stores the measured composition ratio in the memory unit 20 as first information along with the current location of the user at step S220′. In this case, the sensor unit 10 may include a sweat sensor of various sensors because it needs to measure the composition ratio of the sweat. The sweat sensor is a contact type sensor, and is capable of detecting sweat and analyzing a composition ratio. The sweat sensor is capable of a composition ratio of compositions forming the sweat secreted from the user. The sweat sensor is capable of detecting sweat secreted from a user, and of measuring a composition ratio, such as water 99%, sodium 0.4%, chlorine 0.1%, potassium 0.1%, a nitrogen-containing component 0.2%, lactic acid 0.1%, and urea 0.1%. In this case, the composition ratio of sweat is only one embodiment. The composition ratio may be different depending on the current state of the body of a user, and some compositions may have a composition ratio of 0.

The sensor unit 10 stores the composition ratio of the sweat of the user in the memory unit 20 as the first information along with the current location of the user. In this case, the current location of the user may be obtained through various means, and may be obtained through a global positioning system (GPS) module, for example. If the current location of the user has been obtained through the GPS module, the first information may include the composition ratio of the sweat of the user, such as water 99%, sodium 0.4%, chlorine 0.1%, potassium 0.1%, a nitrogen-containing component 0.2%, lactic acid 0.1%, and urea 0.1%, and the current location of the user, such as (43.943°, 4.805°). If the current location of the user is obtained using another means other than the GPS module, it may be included in first information according to the corresponding means.

The number of pieces of the first information stored in the memory unit 20 by the sensor unit 10 may be one or more. For example, after first information including a composition ratio of sweat of a user, such as water 99%, sodium 0.4%, chlorine 0.1%, potassium 0.1%, a nitrogen-containing component 0.2%, lactic acid 0.1%, and urea 0.1%, and the current location of the user, such as (43.943°, 4.805°), is stored, new first information including a composition ratio of sweat of the user, such as water 99%, sodium 0.4%, chlorine 0.2%, potassium 0.1%, a nitrogen-containing component 0.1%, and lactic acid 0.2%, and the current location of the user, such as (22.452°, 7.245°), may be additionally stored. In this case, the existing first information may be deleted, or may be accumulated without being deleted depending on the capacity of the memory unit 20 and stored as new first information. Furthermore, the memory unit 20 may delete existing first information when new first information is stored from the time when first information having a specific amount or more is stored because the capacity of the memory unit 20 may be exceeded if the first information is accumulated and stored. That is, various storage methods may be used by the memory unit 2.

When the first information is stored in the memory unit 20, the communication unit 30 sends the first information, stored in the memory unit 20, to the information providing apparatus at step S230′. In this case, the information providing apparatus is the management server 200 according to a second embodiment of the present invention, and will be described in detail later. The communication unit 30 may send the first information using any one of the known communication methods, such as CDMA, WCDMA, GSM, 3G, and 4G, as described above.

When the communication unit 30 sends the first information to the information providing apparatus, the information providing apparatus generates second information by analyzing the first information. The communication unit 30 receives the second information from the information providing apparatus at step S240′. The control unit 40 may enable the received second information to be stored in the memory unit 20 at step S241′. In this case, the second information may be merged with the location corresponding to the first information, that is, the basis for generating information, or the first information. For example, if the first information has been stored in a location A of the memory unit 20, the second information may be stored in a location A′ of the memory unit 20 or may be stored along with the location A. Furthermore, existing first information may be deleted depending on the capacity of the memory unit 20, and second information may be stored in the location from which the existing first information has been deleted. For example, if first information has been stored in a location A of the memory unit 20, the control unit 40 may delete the first information from the location A by detecting that the communication unit 30 has received second information, and may newly store the second information in the location from which the first information has been deleted. The second information includes information about a commodity to be recommended to the user in connection with the composition ratio of the sweat of the user included in the first information. The second information may include information about the closest place where a commodity to be recommended to the user is handled from the current location of the user. This is described in detail later in connection with the management server 200 according to a second embodiment of the present invention.

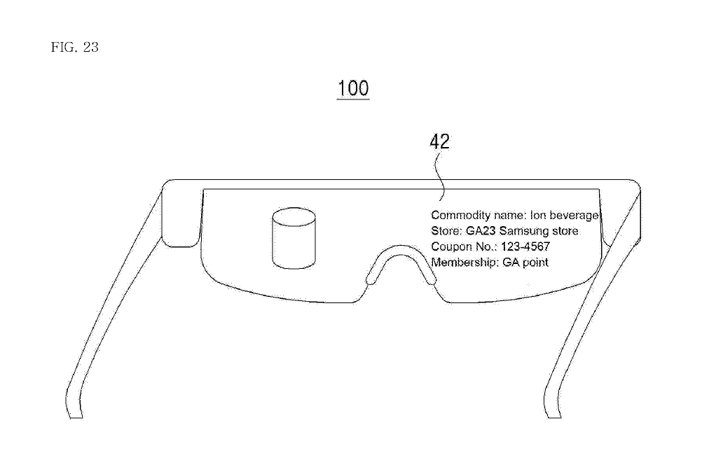

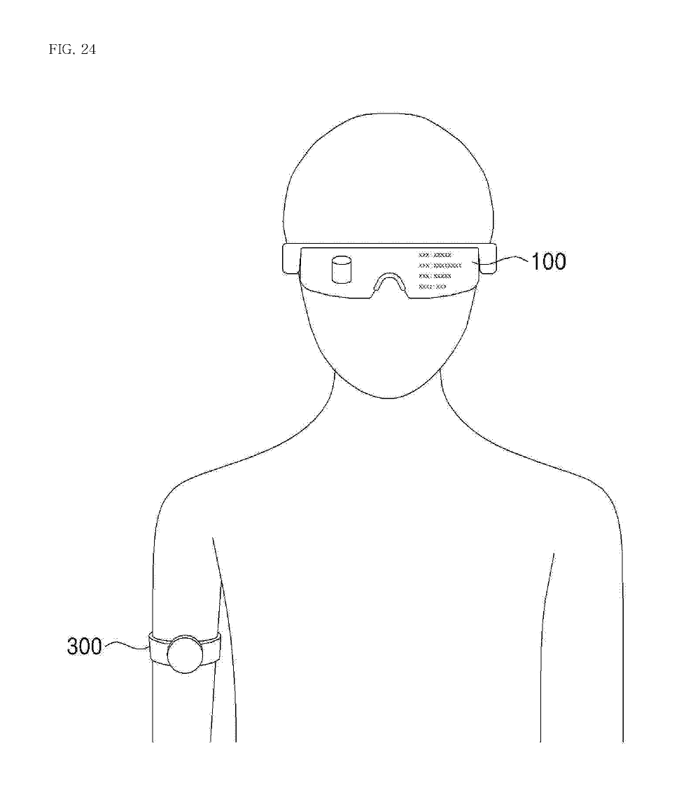

When the communication unit 30 receives the second information, the control unit 40 outputs the second information to one or more of the display unit 42 and the speaker unit at step S250′. From FIG. 23, the state in which the second information has been output through a smart watch can be seen. The second information may be output to the display unit 42 and the speaker unit at the same time. In this case, the wearable device 100 may include both the display unit 42 and the speaker unit. If the wearable device 100 is a smart watch, there is no problem because the wearable device 100 includes both the display unit 42 and the speaker unit. In the case of a wearable device 100 not including both the display unit 42 and the speaker unit, for example, a smart band, etc., there may be a problem in outputting the second information. In this case, the control unit 40 may output the second information through another device capable of being paired with the smart band through specific means, such as Bluetooth. In this case, the type of another device is not limited, and may include any device having visual or auditory output means, such as an earphone, a smart phone, and smart glasses which support a Bluetooth function. From FIG. 24, the state in which the second information is output through smart glasses 100 owned by a user who has worn a smart band 300 can be seen.

A composition ratio of sweat of a user can be analyzed and useful information according to the current state of the body of the user can be provided through smart glasses through steps S210′ to S250′. The smart glasses are one of representative wearable devices which are worn on the wrist of a user and are worn on the location where sweat secreted from the user may be directly detected. Accordingly, the smart glasses correspond to an optimum wearable device 100 in implementing a service providing method according to an embodiment of the present invention. In another embodiment, an example in which the wearable device 100 is smart glasses is described below.

First, the sensor unit 10 detects sweat secreted from the body of a user who has worn the wearable device 100 at step S210″. In this case, the wearable device 100 may include various types of wearable devices. More specifically, the wearable device 100 may include any one of smart glasses, smart glasses, and a smart band. In the following example, the wearable device 100 is assumed to be smart glasses.

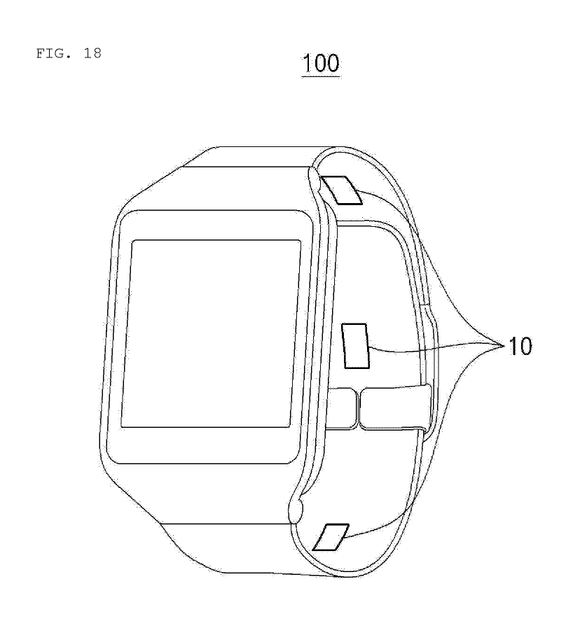

A user wears a smart band functioning as the wearable device 100 on the wrist or back of an arm of the user, and the sensor unit 10 may be mounted on a portion coming into contact with the skin of the user. For example, as shown in FIG. 18, the sensor unit 10 may be mounted on the inside of the strap of the smart band. In this case, there is an advantage in that the sensor unit 10 can directly detect sweat secreted from the wrist or back of an arm of the user. The smart band has a simple configuration compared to other wearable devices, such as a smart watch or smart glasses, etc. The location on which the sensor unit 10 is mounted is limited, but the sensor unit 10 may be mounted on any proper location from which sweat secreted from the wrist or back of an arm of the user can be directly detected in addition to the inside of the strap of the smart band. Furthermore, one or more sensor units 10 may be used. For example, a plurality of the sensor units 10 may be formed on the inside of the smart band. In this case, there is an advantage in that a composition ratio of a user can be measured by more accurately detecting sweat which may be differentially secreted depending on a specific part of the wrist or back of an arm of the user.

When the sensor unit 10 detects the sweat secreted from the body of the user, it measures a composition ratio of the detected sweat and stores the measured composition ratio in the memory unit 20 as first information along with the current location of the user at step S220″. In this case, the sensor unit 10 may include a sweat sensor of various sensors because it needs to measure the composition ratio of the sweat. The sweat sensor is a contact type sensor, and is capable of detecting sweat and analyzing a composition ratio. The sweat sensor is capable of a composition ratio of compositions forming the sweat secreted from the user. The sweat sensor is capable of detecting sweat secreted from a user, and of measuring a composition ratio, such as water 99%, sodium 0.4%, chlorine 0.1%, potassium 0.1%, a nitrogen-containing component 0.2%, lactic acid 0.1%, and urea 0.1%. In this case, the composition ratio of sweat is only one embodiment. The composition ratio may be different depending on the current state of the body of a user, and some compositions may have a composition ratio of 0.

The sensor unit 10 stores the composition ratio of the sweat of the user in the memory unit 20 as the first information along with the current location of the user. In this case, the current location of the user may be obtained through various means, and may be obtained through a global positioning system (GPS) module, for example. If the current location of the user has been obtained through the GPS module, the first information may include the composition ratio of the sweat of the user, such as water 99%, sodium 0.4%, chlorine 0.1%, potassium 0.1%, a nitrogen-containing component 0.2%, lactic acid 0.1%, and urea 0.1%, and the current location of the user, such as (43.943°, 4.805°). If the current location of the user is obtained using another means other than the GPS module, it may be included in first information according to the corresponding means. The smart band may not include a GPS module. In this case, the smart band may receive the current location of the user through another device paired with the smart band, and may store the received current location in the memory unit 20 as the first information.

The number of pieces of the first information stored in the memory unit 20 by the sensor unit 10 may be one or more. For example, after first information including a composition ratio of sweat of a user, such as water 99%, sodium 0.4%, chlorine 0.1%, potassium 0.1%, a nitrogen-containing component 0.2%, lactic acid 0.1%, and urea 0.1%, and the current location of the user, such as (43.943°, 4.805°), is stored, new first information including a composition ratio of sweat of the user, such as water 99%, sodium 0.4%, chlorine 0.2%, potassium 0.1%, a nitrogen-containing component 0.1%, and lactic acid 0.2%, and the current location of the user, such as (22.452°, 7.245°), may be additionally stored. In this case, the existing first information may be deleted, or may be accumulated without being deleted depending on the capacity of the memory unit 20 and stored as new first information. Furthermore, the memory unit 20 may delete existing first information when new first information is stored from the time when first information having a specific amount or more is stored because the capacity of the memory unit 20 may be exceeded if the first information is accumulated and stored. That is, various storage methods may be used by the memory unit 20.

When the first information is stored in the memory unit 20, the communication unit 30 sends the first information, stored in the memory unit 20, to the information providing apparatus at step S230″. In this case, the information providing apparatus is the management server 200 according to a second embodiment of the present invention, and will be described in detail later. The communication unit 30 may send the first information using any one of the known communication methods, such as CDMA, WCDMA, GSM, 3G, and 4G, as described above.

When the communication unit 30 sends the first information to the information providing apparatus, the information providing apparatus generates second information by analyzing the first information. The communication unit 30 receives the second information from the information providing apparatus at step S240″. The control unit 40 may enable the received second information to be stored in the memory unit 20 at step S241″. In this case, the second information may be merged with the location corresponding to the first information, that is, the basis for generating information, or the first information. For example, if the first information has been stored in a location A of the memory unit 20, the second information may be stored in a location A′ of the memory unit 20 or may be stored along with the location A. Furthermore, existing first information may be deleted depending on the capacity of the memory unit 20, and second information may be stored in the location from which the existing first information has been deleted. For example, if first information has been stored in a location A of the memory unit 20, the control unit 40 may delete the first information from the location A by detecting that the communication unit 30 has received second information, and may newly store the second information in the location from which the first information has been deleted. The second information includes information about a commodity to be recommended to the user in connection with the composition ratio of the sweat of the user included in the first information. The second information may include information about the closest place where a commodity to be recommended to the user is handled from the current location of the user. This is described in detail later in connection with the management server 200 according to a second embodiment of the present invention.

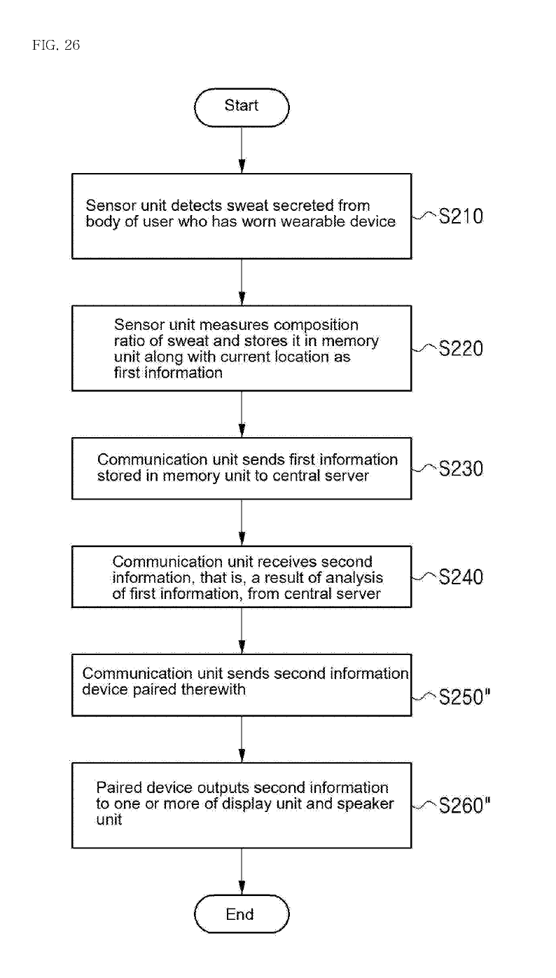

The smart band may not include the display unit 42 and the speaker unit because it has a relatively simple configuration unlike other wearable devices. Accordingly, in steps S250 or S250′ performed through the smart watch and the smart glasses, a method for outputting the second information may be different. The communication unit 30 that has received the second information sends the second information to another device paired with the smart band at step S250″. The paired device outputs the second information to one or more of the display unit 42 and the speaker unit at step S260″. That is, step S260″ may be considered to be step S250 or S250′ performed through the smart watch and the smart glasses. Due to the nature of the smart band, step S250″ for sending the second information to the paired device has been added. FIG. 26 shows a flowchart illustrating a method for providing a customized membership and coupon to a user through a smart band according to a first embodiment of the present invention.

A smart band equipped with the display unit 42 and the speaker unit is recently released. In this case, step S250″ for sending the second information to the paired device may not be performed. The control unit 40 may output the second information to one or more of the display unit 42 and the speaker unit.

The service providing method according to the first embodiment of the present invention, which has been implemented through a smart watch, smart glasses, and a smart band, has been described so far. There are some differences depending on the wearable device 100. A service providing method may include detecting, by the sensor unit 10, sweat, measuring, by the sensor unit 10, a composition ratio of the sweat and storing the measured composition ratio in the memory unit 20 as first information, sending, by the communication unit 30, the first information to the information providing apparatus, receiving, by the communication unit 30, second information, and outputting, by the control unit 40, the second information. Accordingly, information based on the current physical state of a user can be provided. Furthermore, the service providing method according to the first embodiment of the present invention may be installed in the form of a function which is basically provided by a wearable device, more specifically, a smart watch, smart glasses, or a smart band, and may be provided in the form of a downloadable application. In this case, everyone can download and use the function or application if he or she has registered the function or application with an application market regardless of the type of wearable device.

Furthermore, the service providing method according to the first embodiment of the present invention may be implemented in a program form. The program may be stored in a computer-readable recording medium on which a program for being executed on a computer has been recorded or may be distributed through a program providing server.

Hereinafter, the management server 200 according to a second embodiment of the present invention is described.

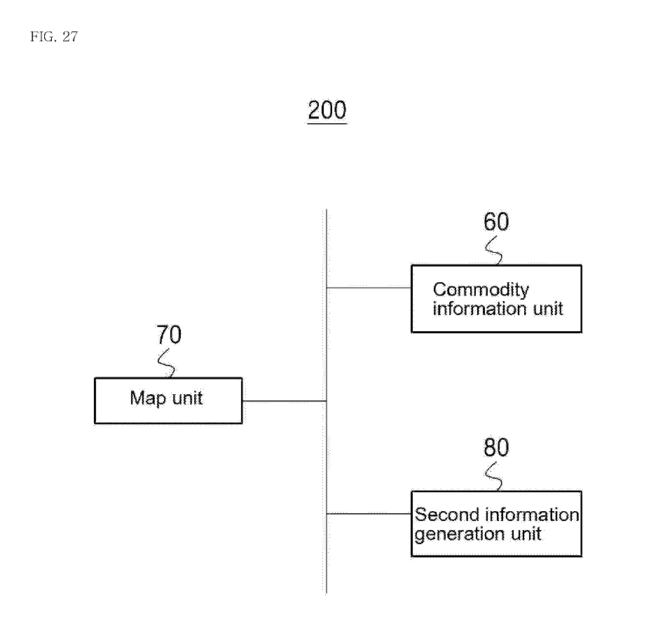

FIG. 27 is a diagram showing the configuration of the management server 200 according to a second embodiment of the present invention. The management server 200 includes a commodity information unit 60, a map unit 70, and a second information generation unit 80, and may further include other elements required to implement an embodiment of the present invention. The management server 200 may be a server for providing corresponding information, for example.

Commodities to be recommended to a user and information about the commodities have been listed and stored in the commodity information unit 60 according to composition that belongs to the compositions of sweat and that has a high composition rate. The compositions of sweat include water of 99% and the remaining sodium (Na), chlorine (Cl), potassium (K), a nitrogen-containing component, lactic acid, urea, etc. (hereinafter referred to as the “remaining compositions”) of 1%. The commodity information unit 60 stores commodities to be recommended to a user and information about the commodities according to composition that belongs to the remaining compositions and that has a high composition ratio. For example, a commodity to be recommended to a user and information about the commodity if the composition ratio of sodium is high, a commodity to be recommended to a user and information about the commodity if the composition ratio of chlorine is high, a commodity to be recommended to a user and information about the commodity if the composition ratio of potassium is high, a commodity to be recommended to a user and information about the commodity if the composition ratio of a nitrogen-containing component is high, a commodity to be recommended to a user and information about the commodity if the composition ratio of lactic acid is high, and a commodity to be recommended to a user and information about the commodity if the composition ratio of urea is high have been listed and stored in the commodity information unit 60. In this case, a commodity to be recommended to a user if the composition ratio of a specific composition is high is a commodity capable of lowering the composition ratio of the specific composition. For example, a commodity to be recommended to a user if the composition ratio of sodium is high may be a commodity capable of lowering the composition ratio of sodium. In an embodiment, commodities to be recommended to a user and information about the commodities may be listed and stored in the commodity information unit 60 according to composition that belongs to the compositions of sweat and that has a low composition ratio. In this case, a commodity to be recommended to a user will be a commodity capable of raising the composition ratio of a corresponding composition. The commodity information unit 60 has limits to information which may be stored therein depending on the size of a storage space. All of commodities to be recommended to a user and information about the commodities according to composition that belongs to the compositions of sweat and that has a high composition rate and commodities to be recommended to a user and information about the commodities according to composition that belongs to the compositions of sweat and that has a low composition ratio may have been listed and stored in the commodity information unit 60.

In the list, priority is not given to a composition having a high composition ratio. If a specific composition of a plurality of the remaining compositions forming sweat has a high composition ratio, a commodity to be recommended to a user and information about the commodity are included in the list. Accordingly, such compositions may be considered to be independent. For example, assuming that a commodity to be recommended to a user and information about the commodity if the composition ratio of sodium is high and a commodity to be recommended to a user and information about the commodity if the composition ratio of chlorine is high have been stored in the list, any one of sodium and chlorine does not need to have a higher composition ratio. The reason for this is that the two compositions are independently handled in the list. The same is true of all of a case where all of commodities to be recommended to a user and information about the commodities according to composition that belongs to the compositions of sweat and that has a low composition ratio are stored in the list, a case where commodities to be recommended to a user and information about the commodities according to composition that belongs to the compositions of sweat and that has a high composition rate are stored in the list, and a case where commodities to be recommended to a user and information about the commodities according to composition that belongs to the compositions of sweat and that has a low composition ratio are stored in the list.

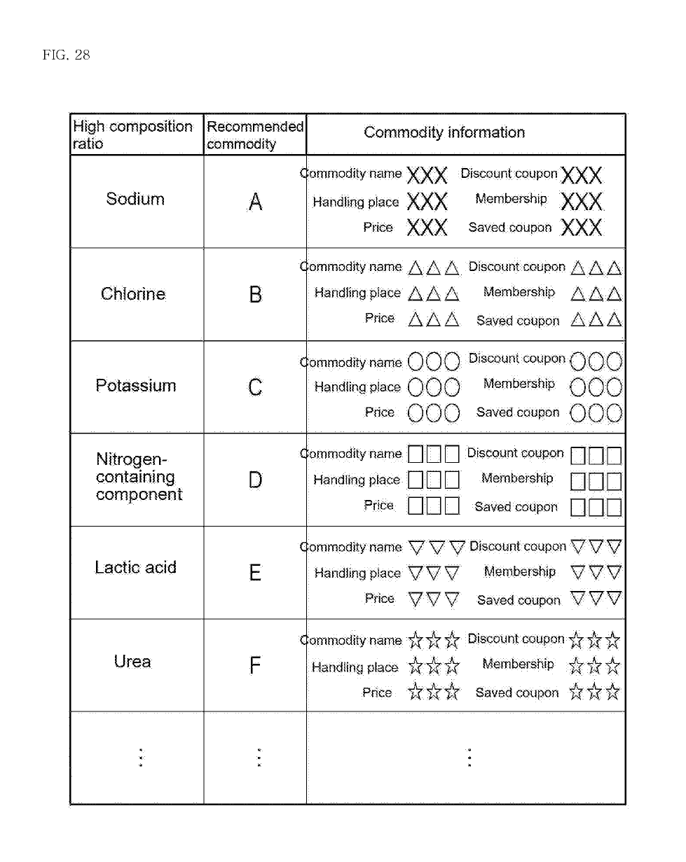

The stored list may be updated. For example, if a commodity to be recommended to a user if the composition ratio of sodium is high is A and information “a” about the commodity A has been stored in the list, a new commodity B and information “b” about the new commodity B may be added. In this case, the existing commodity A and the information “a” about the commodity A may remain intact or may be deleted. From FIG. 28, it may be seen that commodities to be recommended to a user and information about the commodities have been listed and stored according to composition that belongs to the remaining compositions of sweat and that has a high composition ratio.

Information about a commodity includes the name and price of the commodity and the place where the commodity is handled, and may further include one or more of the type of membership point applied, a discount coupon applied, and a saved coupon applied. In this case, the information about a commodity is only one embodiment, and may include all of other pieces of useful information which may help a user to purchase the commodity. Furthermore, a commodity may include any things, such as health adjurvants, which are handled in a drugstore and which may be eaten by a user in addition to food, and the type of commodity is not limited.

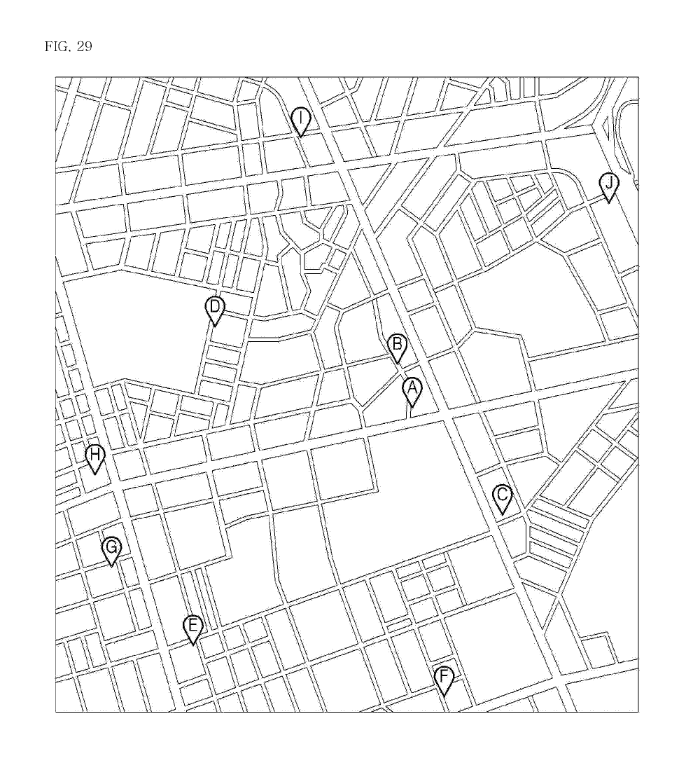

The map unit 70 stores an updatable map. The map stored in the map unit 70 may be a common map provided on a web, but may be a map on which the place where a commodity is handled is indicated in detail. For example, the map may include all of the places where commodities to be recommended to a user are handled, such as a convenience store, a hypermarket, and a department store, as in FIG. 29.

A stored map needs to be updated. The reason for this is that the place where a commodity is handled may be changed. For example, if the location of a convenience store “a” in which a commodity A is handled is changed and the convenience store “a” has been deleted, a corresponding map needs to be modified in order to provide a user with accurate information. Accordingly, the map stored in the map unit 70 needs to be updated, and the update may be performed periodically at specific time intervals or in real time.

The second information generation unit 80 generates second information by analyzing first information received from the communication unit 30 of the wearable device 100. In the description of the service providing method according to the first embodiment of the present invention, the first information has been illustrated as including both the composition ratio of sweat of a user and the current location of the user measured by the sensor unit 10, and the communication unit 30 has been illustrated as receiving second information from the information providing apparatus. In this case, the information providing apparatus is the management server 200 according to the second embodiment of the present invention, and an element configured to generate second information by analyzing first information is the second information generation unit 80. This is described in detail below.

The second information generation unit 80 analyzes first information, and generates second information by selecting one or more pieces of information that belong to information about a commodity stored in the commodity information unit 60 and that comply with the results of the analysis from a list. In this case, the analysis of the first information means that only information about the composition ratio of sweat of a user is extracted from the first information including both the composition ratio of sweat of the user and the current location of the user. For example, if first information includes water 99%, sodium 0.4%, chlorine 0.1%, potassium 0.1%, a nitrogen-containing component 0.2%, lactic acid 0.1%, and urea 0.1%, that is, the composition ratio of sweat of a user, and information about the current location (43.943°, 4.805°) of the user, the second information generation unit 80 may extract only water 99%, sodium 0.4%, chlorine 0.1%, potassium 0.1%, a nitrogen-containing component 0.2%, lactic acid 0.1%, and urea 0.1%, that is, the composition ratio of sweat of the user, from the first information. The second information generation unit 80 selects one or more pieces of information that belong to information about a commodity stored in the commodity information unit 60 and that comply with the results of analysis from a list based on information extracted from first information. In the selection, a specific comparison procedure is necessary. In this case, how the information is selected from the list according to what criterion matters. Accordingly, information about the composition ratio of sweat in a normal physical state is previously stored in the second information generation unit 80. For example, if the composition ratio of sweat of a user, including water 99%, sodium 0.2%, chlorine 0.1%, potassium 0.2%, a nitrogen-containing component 0.2%, lactic acid 0.1%, and urea 0.2% in a normal physical state, has been stored in the second information generation unit 80, the second information generation unit 80 may detect that the composition ratio of sodium is high and the composition ratios of potassium and urea are low by comparing the composition ratio of sweat, including water 99%, sodium 0.2%, chlorine 0.1%, potassium 0.2%, a nitrogen-containing component 0.2%, lactic acid 0.1%, and urea 0.2% in the normal physical state, with the composition ratio of sweat of a user having water 99%, sodium 0.4%, chlorine 0.1%, potassium 0.1%, a nitrogen-containing component 0.2%, lactic acid 0.1%, and urea 0.1%, that is, information extracted from the first information. The composition ratio of sweat in a normal physical state may have been stored in a specific value range not a specific value as in the above embodiment. For example, the composition ratio of sweat in the normal physical state may be stored in a specific value range, for example, water 99%, sodium of 0.1 to 0.2%, chlorine of 0.1 to 0.2%, potassium of 0.1 to 0.2%, a nitrogen-containing component of 0.05 to 0.2%, lactic acid of 0.1 to 0.2%, urea 0.1 to 0.2%, etc. In this case, the second information generation unit 80 may detect information about a composition that belongs to the composition ratio of sweat of a user and that deviates from a specific value range.

After detecting a composition of sweat, that is, a difference according to the results of a comparison between the composition ratio of sweat of a user previously stored in a normal physical state and the composition ratio of sweat of the user extracted from the first information, the second information generation unit 80 generates second information by selecting one or more pieces of information that belong to information about a commodity stored in the commodity information unit 60 and that comply with the results of the analysis. As described above, commodities to be recommended to a user and information about the commodities have been stored in the commodity information unit 60 according to composition that belongs to the remaining compositions other than water and that has a high composition ratio. For example, a commodity to be recommended to a user and information about the commodity if the composition ratio of sodium is high, a commodity to be recommended to a user and information about the commodity if the composition ratio of chlorine is high, a commodity to be recommended to a user and information about the commodity if the composition ratio of potassium is high, a commodity to be recommended to a user and information about the commodity if the composition ratio of a nitrogen-containing component is high, a commodity to be recommended to a user and information about the commodity if the composition ratio of lactic acid is high, and a commodity to be recommended to a user and information about the commodity if the composition ratio of urea is high have been listed and stored in the commodity information unit 60. Alternatively, commodities to be recommended to a user and information about the commodities may be listed and stored in the commodity information unit 60 according to composition having a low composition ratio. Accordingly, in the above embodiment, if the second information generation unit 80 detects that the composition ratio of sodium is high and the composition ratios of potassium and urea are low, it may select a commodity to be recommended to the user and information about the commodity if the composition ratio of sodium is high, a commodity to be recommended to the user and information about the commodity if the composition ratio of potassium is low, and a commodity to be recommended to the user and information about the commodity if the composition ratio of urea is low from the list. In this case, commodities and information about the commodities selected from the list by the second information generation unit 80 may include a commodity capable of lowering the composition ratio of sodium and information about the commodity, a commodity capable of raising the composition ratio of potassium and information about the commodity, and a commodity capable of raising the composition ratio of urea and information about the commodity. The information about a commodity includes the name and price of the commodity and the place where the commodity is handled, and may include one or more of the type of membership point applied, a discount coupon applied, and a saved coupon applied as described above. A kind of marketing effect can be obtained by providing a user with second information including such information.

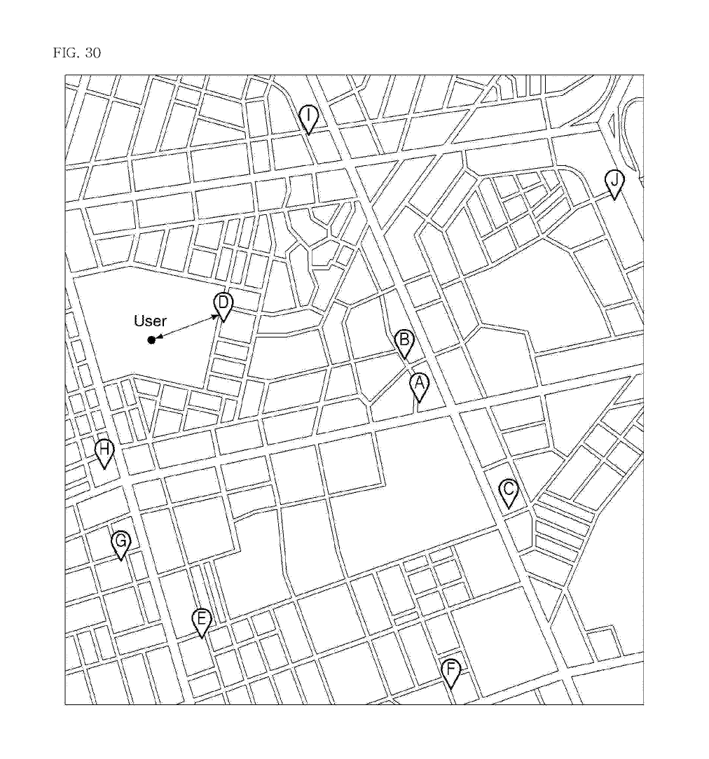

The second information generation unit 80 may generate second information by selecting the places where one or more commodities selected from a list are handled, included in information about the commodities, from the map unit 70. For example, in the above embodiment, if it is detected that the composition ratio of sodium is high and the composition ratios of potassium and urea are low, the second information generation unit 80 may select the place “a” where a commodity A to be recommended to a user is handled if the composition ratio of sodium is high, the place “b” where a commodity B to be recommended to a user is handled if the composition ratio of potassium is low, and the place “c” where a commodity C to be recommended to a user is handled if the composition ratio of urea is low from the map unit. In this case, the places “a”, “b”, and “c” are the places where commodities included in information about the commodities are handled. Furthermore, the place where a commodity is handled, selected by the second information generation unit 80, corresponds to the place that is the closest to the current location of a user. The first information includes the current location of a user in addition to the composition ratio of sweat of the user, and thus a kind of convenience can be improved by providing the place where a commodity is handled and which is the closest to the current location of the user. For example, if the places where a commodity A to be recommended to a user is handled if the composition ratio of sodium is high are “a”, “b”, and “c”, the second information generation unit 80 may extract the current location of the user from first information and select the closest place of the places “a”, “b”, and “c.” FIG. 30 shows an example in which the place where a commodity is handled, which is the closest to the current location of a user, is selected.

A commodity capable of maintaining the homeostasis of the physical state of a user and information about the closest place where the corresponding commodity is handled can be recommended to the user who has worn the wearable device 100 through the management server 200. Furthermore, the user may also be additionally provided with information one or more of the type of membership point, a discount coupon, and a saved coupon which are helpful to purchase the commodity in addition to the information about the recommended commodity. Accordingly, a double purpose can be obtained. FIG. 30 shows an example in which second information is displayed on the display unit 42 of a smart watch, that is, the wearable device 100 of a user.

The service providing method according to the first embodiment of the present invention may be implemented in the form of an application (hereinafter referred to as a “first application”) as described above, and may provide a better service to a user while operating in conjunction with another application (hereinafter referred to as a “second application”). For example, when second information is output to the wearable device 100 through a service providing method implemented in the form of the first application, a user may purchase a recommended commodity through the second application operating in conjunction with the first application even without a need to visit the place where the recommended commodity is handled. More specifically, if a commodity recommended to a user is coffee sold in a coffee shop, the user may check information about the commodity, the place where the commodity is handled, a membership point, a discount coupon, a saved coupon, etc. through the first application, and may pay the commodity in advance through the second application. In this case, the user does not need to visit a corresponding place where the commodity is handled and to pay the commodity, and may receive the commodity as soon as the user visits the place, thereby being capable of improving user convenience. Furthermore, a saved membership point, a discount coupon, and a saved coupon providing service may be provided in the second application itself in order to obtain a separate marketing and customer attraction effect.

Technological characteristics described in this specification and an implementation for executing the technological characteristics may be implemented using a digital electronic circuit, may be implemented using computer software, firmware or hardware including the structure described in this specification and structural equivalents thereof, or may be implemented using a combination of one or more of them. Furthermore, the implementation for executing the technological characteristics described in this specification may be implemented using a computer program product, that is, a module regarding computer program instructions encoded on a kind of program storage media in order to control the operation of a processing system or for execution by the processing system.

A computer-readable medium may be a machine-readable storage device, a machine-readable storage substrate, a memory device, a composition of materials that affect a machine-readable electromagnetic signal or a combination of one or more of them.

In this specification, the terms “apparatus or device” or “system” cover all apparatuses, devices, and machines for processing data, for example, including a processor, a computer or a multi-processor, or a computer. The processing system may include, for example, code that forms processor firmware, a protocol stack, a database management system, an operating system, or all types of code that form an execution environment for a computer program when a combination of one or more of them is requested, in addition to hardware.

A computer program also known as a program, software, a software application, a script or code may be written in any form of a programming language which includes a compiled or interpreted language or a transcendental and/or procedural language, and may also be implemented in any form including an independent program or module, a component, a subroutine or other units suitable for being used in a computer environment.

The computer program does not need to necessarily correspond to a file of a file system. The program may be stored in a single file provided to a requested program, multiple files that interact with each other (e.g., a file that stores one or more modules, a lower program or part of code), or another program or part of a file including data (e.g., one or more scripts stored in markup language document).

The computer program may be located in a single site or distributed to a plurality of sites and may be implemented to be executed on multiple computers or one or more computers interconnected over wired/wireless communication networks.

A computer-readable medium suitable for storing computer program instructions and data may include semiconductor memory devices, such as EPROM, EEPROM, and a flash memory device, for example, all types of non-volatile memory, media, and memory devices including magnetic disks, such as an internal hard disk or an external disk, magneto optical disks, CDs, and DVDs. The processor and the memory may be supplemented by a logic circuit for a special object or may be integrated into the logic circuit for a special object.

An implementation for executing the subject matter described in this specification may be implemented in an operation system including a backend component, such as a data server, a middleware component, such as an application server, a frontend component, such as a client computer having a web browser or graphic user interface capable of interacting with the implementation of the subject matter described by a user in this specification or all combinations of one or more of the backend, middleware, and frontend components. The component of the system may be accessed by any type or medium for digital data communication, such as a communication network.

Hereinafter, more detailed embodiments capable of implementing the elements included in the wearable device and method for providing a service according to the measurement of a blood alcohol level and the management server therefor, described in this specification, along with the aforementioned contents are described in detail.

The wearable device and method for providing a service according to the measurement of a blood alcohol level and the management server therefor, which have been described in this specification, may be used partially or generally through a server related to a client device or web-based storage system or means for executing computer software, program code or instructions on one or more processors included in a server. In this case, the processor may be part of a server, a client, network infrastructure, or a computing platform, such as a mobile computing platform or fixed computing platform. More specifically, the processor may be a kind of computer or processing device capable of executing program instructions, code, etc. Furthermore, the processor may further include memory for storing the method, instructions, code or program for providing a service according to the measurement of a blood alcohol level. If memory is not included in the processor, the processor may access a storage device, such as CD-ROM, DVD, memory, a hard disk, a flash drive, RAM, ROM, or a cache in which the method, instructions, code or program for providing a service according to the measurement of a blood alcohol level.

Furthermore, the wearable device and method for providing a service according to the measurement of a blood alcohol level, which have been described in this specification, may be used partially or generally through an apparatus for executing computer software on a server, a client, a gateway, a hub, a router or network hardware. In this case, the software may be executed in various types of servers, such as a file server, a print server, a domain server, an Internet server, an intranet server, a host server, and a distributed server. The aforementioned servers may further include memory, a processor, a computer-readable storage medium, a storage medium, a communication device, a port, a client, and an interface capable of accessing other servers over wired/wireless networks.

Furthermore, the method, instructions, or code for providing a service according to the measurement of a blood alcohol level may also be executed by a server. Other devices required to execute the method for providing a service according to the measurement of a blood alcohol level may be implemented as part of a hierarchical structure associated with the server.

Furthermore, the server may provide an interface to other devices including a client, another server, a printer, a database server, a print server, a file server, communication a server, and a distributed server without limitation. A connection through the interface may enable a program to be easily executed at a remote place over wired/wireless networks.

Furthermore, any one of devices connected to the server through the interface may further include at least one storage device capable of storing the method, instructions or code for providing a service according to the measurement of a blood alcohol level. The central processor of the server may provide instructions, code, etc. to be executed on another device to the device so that the instructions, code, etc. are stored in a storage device.

The wearable device and method for providing a service according to the measurement of a blood alcohol level, which have been described in this specification, may be used partially or generally through network infrastructure. In this case, the network infrastructure may include all of devices, such as a computing device, a server, a router, a hub, a firewall, a client, a personal computer, a communication device, and a routing device, and separate modules capable of executing respective functions. The network infrastructure may further include storage media, such as story flash memory, a buffer, a stack, RAM, and ROM, in addition to the aforementioned devices and module. Furthermore, the method, instructions or code for providing a service according to the measurement of a blood alcohol level may also be executed by and stored in any one of the device, module, and storage medium included in the network infrastructure. Another device required to execute the method for providing a service according to the measurement of a blood alcohol level may also be implemented as part of the network infrastructure.

Furthermore, the wearable device and method for providing a service according to the measurement of a blood alcohol level, which have been described in this specification, may be implemented using hardware or hardware suitable for a specific application and software. In this case, the hardware includes all of general-purpose computer devices, such as a personal computer and a mobile communication terminal, and a business type specific computer device. The computer device may be implemented using a device, such as memory, a microprocessor, a microcontroller, a digital signal processor, an application-specific integrated circuit, a programmable gate array, programmable array logic or a combination of them.

The aforementioned computer software, instructions, code, etc. may be stored or accessed by a readable device. In this case, the readable device may include memory, such as a computer component including digital data used for computing for a specific time, semiconductor storage, such as RAM or ROM, permanent storage, such as an optical disk, high-capacity storage, such as a hard disk, a tape and a drum, optical storage, such as a CD or DVD, and network access type storage, such as flash memory, a floppy disk, a magnetic tape, a paper tape, an independent type RAM disk, high-capacity storage detachable from a computer, dynamic memory, static memory, variable storage, and cloud. In this case, the instructions, code, etc. Include all of languages, such as data-oriented languages, such as SQL and dBase, system languages, such as C, Objective C, C++, and Assembly, architecture languages, such as Java and NET, and application languages, such as PHP, Ruby, Perl, and Python, but are not limited thereto. The instructions, code, etc. may include all of languages widely known to those skilled in the art to which the present invention pertains.

Furthermore, the “computer-readable medium” described in this specification includes all of media which contribute to the provision of instruction to a processor in order to execute a program. More specifically, the “computer-readable medium” includes non-volatile media, such as a data storage device, an optical disk and a magnetic disk, volatile media, such as dynamic memory, and transmission media, such as a coaxial cable, a copper wire and an optical fiber for sending data, but is not limited thereto.

The elements for executing the technical characteristics of the present invention included in the block diagrams and flowcharts shown in the accompanying drawings of this specification mean the logical boundary between the elements.

In accordance with a software or hardware embodiment, however, the functions of the illustrated elements and functions thereof may be implemented so that the elements and functions thereof are executed in the form of an independent software module, a monolithic software structure, code, a service or a combination of them and are stored in a medium which is executable by a computer including a processor capable of executing stored program code and instructions. Accordingly, all of such embodiments should be construed as belonging to the scope of the present invention.

Accordingly, the accompanying drawings and technologies thereof describe the technical characteristics of the present invention, but should not be simply reasoned unless a specific array of software for implementing such technical characteristics is clearly described otherwise. That is, the aforementioned various embodiments may be present and may be partially modified while having the same technical characteristics as those of the present invention. Accordingly, such modified embodiments should be construed as belonging to the scope of the present invention.

Furthermore, the flowchart describes operations in the drawing in a specific sequence, but has been illustrated to obtain the most preferred results. It should not be understood that such operations must be executed or all the illustrated operations must be executed in the illustrated specific sequence or sequential order. In a specific case, multi-tasking and parallel processing may be advantageous. Furthermore, the separation of various system components in the aforementioned embodiments should not be construed as requesting such separation in all the embodiments. It should be understood that the aforementioned program components and systems may be integrated into a single software product or packaged into a multi-software product.

In an embodiment of the present invention, a user can refrain from overdrinking because a blood alcohol level of the user is measured in real time and thus he or she can continue to check the blood alcohol level.

Furthermore, in an embodiment of the present invention, a coupon and points available for a hangover after drinking can be provided to a user.

Furthermore, in an embodiment of the present invention, a chauffeur service can be provided to a user although the user does not call the chauffeur service because the management server checks the time when the user performs payment at a food store along with a blood alcohol level of the user and checks the time when the chauffeur service is required.

In an embodiment of the present invention, an accurate quantity of motion of a user can be measured by analyzing the quantity of motion by taking into consideration body information, such as the age, sex, height, weight and body composition of the user in addition to a motion of the user in measuring the user's quantity of motion.

Furthermore, in an embodiment of the present invention, fitness equipment information, sporting goods information, food information and a coupon which enable a user to have a required amount of muscle, body fat, etc. can be provided based on information, such as the body composition, quantity of motion, etc. of the user.

Furthermore, in an embodiment of the present invention, a user can conveniently and frequently perform self-diagnosis without a burden regardless of people of all ages and both sexes by conveniently measuring his or her body composition for himself or herself.

Furthermore, an embodiment of the present invention can help a user to have a balanced body by checking the amount of activity of each part, performing a comparison between the amounts of muscle and body fat of parts, and notifying the user of an unbalanced part.

In accordance with an embodiment of the present invention, there is an advantage in that a commodity capable of maintaining the homeostasis of the body of a user and information about the commodity can be provided to the user by measuring the composition ratio of sweat of the user.

Furthermore, information about a commodity provided to a user includes one or more of the type of membership point applied, a discount coupon applied, and a saved coupon applied. A user can be induced to purchase a corresponding commodity, and a company that provides a service can obtain a kind of marketing effect.

As described above, this specification is not intended to limit the embodiments of the present invention by the proposed detailed terms. Accordingly, although the present invention has been described in detail in connection with the aforementioned embodiments, a person having ordinary skill in the art to which the present invention pertains may alter, change, and modify the embodiments without departing from the scope of the present invention.

Advantages of the present invention are not limited to the aforementioned advantages and may include various other advantages within a range evident to those skilled in the art from the following description.

The scope of the present invention is defined by the appended claims rather than the detailed description, and the present invention should be construed as covering all modifications or variations derived from the meaning and scope of the appended claims and their equivalents.

What is claimed is:

1. A wearable device, comprising:
an alcohol concentration measurement unit configured to measure a blood alcohol level of a user in a predetermined cycle;
a location information measurement unit configured to measure information about a location of the user;
a coupon request unit configured to request information about a coupon or point capable of being used at a store within a predetermined range from a management server when the blood alcohol level of the user is a predetermined value or more; and
a communication unit configured to transmit and receive information about the blood alcohol level of the user and information about the location of the user to and from an external apparatus over a communication network.

2. The wearable device of claim 1, wherein the alcohol concentration measurement unit measures the blood alcohol level based on a change of a body temperature or heart rate of the user.

3. The wearable device of claim 1, wherein the alcohol concentration measurement unit stores a blood alcohol level of the user, checks a drinking habit of the user, and notifies the user of a possibility of a disease attributable to the drinking habit of the user.

4. The wearable device of claim 1, further comprising a chauffeur request unit configured to query the user as to whether a chauffeur call request is to be transmitted to the management server when the blood alcohol level of the user is the predetermined value or more.

5. The wearable device of claim 1, wherein when the alcohol concentration of the user is the predetermined value or more, the communication unit sends a settlement signal of the user to the management server when the user performs settlement at a food store.

6. The wearable device of claim 1, further comprising a display unit configured to display the blood alcohol level of the user in real time and to display a warning message when the blood alcohol level of the user is the predetermined value or more.

7. A wearable device, comprising:
a measurement module configured to measure a body composition or motion of a user;
a control module configured to calculate a quantity of motion based on the measured motion of the user; and
a communication module configured to send information about the measured body composition or quantity of motion to a management server and to receive feedback information for the transmitted information from the management server.

8. The wearable device of claim 7, wherein the measurement module comprises:
a body composition measurement sensor configured to measure the body composition by measuring a resistance value generated when an electric current flows into a body of the user; and
a motion sensor configured to measure at least one of a muscle motion, heartbeat, and respiration volume of the user.

9. The wearable device of claim 7, wherein the measurement module measures an amount of muscle or body fat of each part of the user and notifies the user of an unbalanced part.

10. The wearable device of claim 7, wherein the control module calculates the quantity of motion by taking into consideration at least one of pieces of body information comprising a height, weight, body composition, and sex of the user.

11. The wearable device of claim 7, wherein the feedback information comprises at least one of fitness equipment information, sporting goods information, food information, and coupon information corresponding to information about a body of the user.

12. A service providing method, comprising:
(a) detecting, by a sensor unit, sweat secreted from a body of a user who has worn a wearable device;
(b) measuring, by the sensor unit, a composition ratio of the detected sweat and storing the measured composition ratio in a memory unit along with a current location of the user as first information;
(c) sending, by a communication unit, the first information stored in the memory unit to an information providing apparatus;
(d) receiving, by the communication unit, second information which is a result of an analysis of the first information from the information providing apparatus; and
(e) outputting, by a control unit, the second information to one or more of a display unit and a speaker unit.

13. The service providing method of claim 12, wherein the wearable device comprises any one of a smart watch, smart glasses, and a smart band.

14. The service providing method of claim 13, wherein if the wearable device is the smart watch, the sensor unit is mounted on one or more of a back of a main body of the smart watch and an inside of a strap of the smart watch.

15. The service providing method of claim 13, wherein if the wearable device is the smart glasses, the sensor unit is mounted on one or more of a nose pad, inside of a temple, and inside of an end piece of the smart glasses.

16. The service providing method of claim 12, wherein the second information comprises information about a commodity to be recommended to the user in relation to the composition ratio of the sweat of the user which is included in the first information.

17. The service providing method of claim 16, wherein the second information further comprises information about a place where a commodity to be recommended to the user is handled, which is a closest to the current location of the user.
